(19) United States
(12) Patent Application Publication
(10) Pub. No.: US 2022/0084660 A1
(43) Pub. Date: Mar. 17, 2022

(54) ARTIFICIAL INTELLIGENCE PROCESSING SYSTEM AND AUTOMATED PRE-DIAGNOSTIC WORKFLOW FOR DIGITAL PATHOLOGY

(71) Applicant: Leica Biosystems Imaging Inc., Vista, CA (US)

(72) Inventors: Walter Georgescu, Vista, CA (US); Kiran Saligrama, Vista, CA (US); Carlos Luna, Vista, CA (US); Darragh Lawler, Vista, CA (US); Claude Lacey, Vista, CA (US)

(21) Appl. No.: 17/416,376

(22) PCT Filed: May 29, 2020
(86) PCT No.: PCT/US2020/035342

§ 371 Date: Jun. 18, 2021

Related U.S. Application Data
(60) Provisional application No. 62/854,030, filed on May 29, 2019.
(60) Provisional application No. 62/854,110, filed on May 29, 2019.

(51) Int. Cl. G16H 30/40; G16H 30/20; G16H 50/20; G16H 10/60; G06N 3/08

(52) U.S. Cl. CPC G16H 30/40; G16H 30/20; G06N 3/08; G16H 10/60; G16H 50/20

(57) ABSTRACT

A digital pathology system comprising an AI processing module configured to invoke an instance of an AI processing application for processing image data from a histological image and an application module configured to invoke an instance of an application operable to perform an image processing task on a histological image associated with a patient record, wherein the image processing task includes an AI element. The application creates processing jobs to handle the AI elements of its task which are handled by the AI processing module. The AI processing module may be a CNN that processes a histological image to identify tumors by classifying image pixels into one of multiple tissue classes of tumorous or non-tumorous tissue. A test ordering module automatically determines based on identified tissue classes whether additional tests should be performed on the tissue sample. For each additional test, an order is automatically created and submitted. Advantageously, upon first review by a pathologist, the patient record includes the histological image and results from the automatically ordered additional tests.

### BACKGROUND

### Field of the Invention

The present disclosure generally relates to a distributed artificial intelligence (“AI”) processing system for processing digital pathology data and more particularly relates to an automated pre-diagnostic workflow for acquiring and processing image data from biological tissue samples prior to diagnostic assessment.

### Related Art

In the field of digital pathology, convolution neural networks (CNN) and other artificial intelligence processing techniques are of interest for image processing of histological images of breast cancer and other cancers, as stored as whole slide images (WSIs) in a virtual slide. In principle, automated AI processing methods for analyzing histological images and identifying tumors should be much faster than manual outlining and be capable of more accurate and reproducible results. AI and CNN processing capability may be hosted in and delivered in a distributed network, such as in a cloud-computing environment. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. It is therefore an interesting challenge to adapt such a model of service in the field of digital pathology for processing histological image data.

Image analysis of a biological tissue sample, e.g. from a biopsy, typically involves slicing the tissue sample into multiple adjacent thin cross-sections, referred to as serial sections, to visualize structures of interest within the tissue sample. The serial sections are typically mounted on respective microscope slides. Visual analysis of mounted serial sections can be carried out by the naked eye (grossly) and in more detail by traditional or digital microscopy. Coherent, i.e. successive, serial sections of a tissue sample are typically cross-compared by histologists and pathologists, as well as other relevant health professionals, to identify and locate the same tissue structure through the serial sections. Each serial section is stained with a different stain, each stain having a different tissue affinity to highlight different cells of different tissue types, or different cell features. For example, pathologists often cross compare serial sections that have been variously stained to aid in identifying and locating tissue structures of interest, such as groups of cancer cells or pre-cancerous cells that form tumors.

The traditional way for pathologists to examine a slide is to observe a glass slide under a microscope. The pathologist will start by viewing the slide with a low magnification objective. When an area with potential diagnostic value is observed, the pathologist will switch to a high magnification objective to look in more detail at that area. Subsequently, the pathologist will switch back to low magnification to continue examining other areas on the slide. This low-high-low magnification viewing sequence may be repeated several times over the slide, or set of slides from serial sections, until a definite and complete diagnosis can be made for the slide tissue sample.

In the past twenty years, the introduction of digital scanners has changed this traditional workflow (Molin et al 2015). A digital scanner can acquire an image of an entire glass slide, a so-called whole slide image (WSI), and save it as a digital image data file in a largely automated process that does not need a pathologist. The resulting image data file is typically stored in a slide database from where it is available via a clinical network to a pathologist at a viewing workstation with a high-resolution display, the workstation having a visualization application for this purpose. The pathologist therefore no longer needs to work at a microscope, but rather accesses pre-scanned images from a slide database over a clinical network.

A widely used diagnostic approach stains a first serial section of a tissue sample with hematoxylin and eosin, referred to as H&E staining, where hematoxylin and eosin stain tissue in a complementary manner. Namely, hematoxylin has a relatively high affinity for nuclei, while eosin has a relatively high affinity for cytoplasm. H&E stained tissue gives the pathologist important morphological and positional information about the tissue. For example, typical H&E staining colors nuclei blue-black, cytoplasm varying shades of pink, muscle fibers deep pinky red, fibrin deep pink, and red blood cells orange/red. The pathologist uses the positional (e.g., color) information derived from the H&E stained tissue to estimate the location of corresponding tissue regions on successive serial sections of the tissue that are typically immunohistochemically (IHC) stained with specific markers to color cancerous and pre-cancerous cells. Taking the example of breast cancer, based on the expression of certain genes, the tissue types involved in a breast cancer can be divided into different molecular subtypes. A commonly used classification scheme is as follows:

1. Luminal A: ER+, PR+, HER2−

2. Luminal B: ER+, PR−, HER2+

3. Triple-Negative Breast Cancer (TNBC): ER−, PR−, HER2−

4. HER2-enriched: HER2+, ER−, PR−

5. Normal-like.

ER stands for estrogen receptor. PR stands for progesterone receptor. HER2 stands for human epidermal growth factor receptor 2. IHC stains specific to expression of the above genes have been developed including by way of example: HER2 protein (a membrane-specific marker), Ki67 protein (a nuclei-specific marker), and ER and PR markers.

One widely practiced workflow is for pathologists to use H&E staining to perform initial diagnosis on tissue that is suspected to be cancerous. If the H&E stained section reveals cancerous tissue, the pathologist may order additional tests, where the specific additional tests selected by the pathologist will depend on the type of cancer present. For example, if a pathologist detects invasive breast cancer in an H&E slide, he or she may order an HER2 stain to determine if the cancer may be treated with drugs such as herceptin that target the HER2 receptor (Wolff et al. 2013). Based on a provisional diagnosis of a specific type of cancer or cancers from the H&E stained image, there will be a standard protocol which specifies which additional tests should be performed, these tests including, but not being confined to, staining further serial sections with the relevant markers and obtaining histological images from those serial sections. Once these additional test results are available, the pathologist will then review the newly available images from the additional tests stains alongside the H&E stained image and make a diagnosis.

Therefore, what are needed are systems and methods that overcome these significant problems found in the conventional systems as described above.

### SUMMARY

According to one aspect of the disclosure, there is provided a distributed digital pathology system. The system comprises an artificial intelligence processing module configured to invoke an instance of an artificial intelligence processing application for processing image data from a histological image or part thereof. The system further comprises an application module operatively connectable to the artificial intelligence processing module and configured to invoke an instance of an application operable to perform an image processing task on a histological image associated with a patient record, wherein the image processing task includes an artificial intelligence element, the application instances being configured to create processing jobs to handle the artificial intelligence elements, to send these jobs to the artificial intelligence processing instances for processing, and to receive back processing results from the artificial intelligence processing module. The system may further comprise a data repository configured to store records of patient data including histological images or sets thereof, and operatively connectable to the application module to exchange patient data, such as a virtual slide library or database.

In some embodiments, the artificial intelligence processing module is configured with a data retention policy which promptly and permanently deletes the image data contained in the processing jobs it receives as soon as practically possible after processing the image data is complete. Moreover, the image data may be broken up into sub-units of tiles which may be sent sequentially or non-sequentially from the application module to the artificial intelligence processing module, for example using a packet-based communication protocol. If the artificial intelligence processing instances are configured to process image data in units of patches, as is likely for certain CNN algorithms, the image data can be supplied to the artificial intelligence processing module from the application module in units of tiles which map to the patches. For example, there may be a one-to-one mapping or many-to-one mapping between tiles and patches, or some more complex mapping, e.g. to provide overlapping margins between adjacent patches. The data retention policy is configured to promptly and permanently delete the image data contained in the processing jobs patchwise or tilewise as soon as practically possible after processing each patch or tile. In addition, image tiles may be shuffled with tiles from other images by the application module so that what would appear to be a complete image to a third party snooping the communication channel between application module and artificial intelligence processing module is in fact not.

In some embodiments, the patient data includes additionally metadata linking patient identity to the image data such that when the image data is detached from the metadata the image data is anonymous. The communication between the data repository and the application module may be configured such that the metadata linking the image data to a patient identity is retained in the data repository and not sent to the application module in fulfillment of the processing jobs, so the image data received by the application module is anonymized.

Metadata that could be retained by the data repository and not sent to the application module may include, the slide's barcode, the macro image (i.e. the low-resolution image of the whole slide which serves for orientation of high-resolution tiles), image data relating to non-tissue areas of the slide. Withholding this metadata from the application module would make it extremely hard to extract any information that would enable patient identity to be deduced.

Through these measures, the data becomes less and less identifiable as it moves from the data repository, which may for example be a virtual slide library, to the application module and then on to the artificial intelligence processing module. The data repository has access to all the information, the application module only receives the image data and some other parameters derived from, but not revealing, the patient data, and the artificial intelligence processing module only receives image tiles whose information content may be further obfuscated, e.g. by shuffling with tiles from other images or by making sure that at any one moment in time only a small subset of the image data exists in the artificial intelligence processing module and in transit between the application module and the artificial intelligence processing module.

In some embodiments, the artificial intelligence processing module includes a statistics collection unit operable to monitor and record processing of the artificial intelligence elements.

An artificial intelligence processing configuration module may be provided, this module having a user interface and an interface with the artificial intelligence processing module, thereby to enable a user to configure artificial intelligence processing resource in the artificial intelligence processing module.

The application module may further include an image processing task allocator operable to decide on allocation of artificial intelligence elements of image processing tasks between performance internally in the application module and performance by the artificial intelligence processing module with a processing job. Where the artificial intelligence task, e.g. a machine learning classifier, is executed can then be flexibly decided upon, so may be run on the local machine, on a virtual machine or through an application programming interface (API) that abstracts the hardware altogether such as Azure functions. These decisions may be based on user setup and preferences as well as automated estimates of processing power needed for the task execution of any particular processing job and availability and loading of the different computing resources.

The artificial intelligence processing may be based on a convolutional neural network. The convolutional neural network may be a fully convolutional neural network. For example, the convolutional neural network may be configured to identify tumors in image data from the histological images.

According to another aspect of the disclosure, there is provided a digital pathology image processing method comprising: receiving a request to perform image processing on a histological image associated with a patient record, and, in response thereto; invoking an instance of an application operable to perform an image processing task on the histological image, wherein the image processing task includes an element of artificial intelligence; creating a processing job for an artificial intelligence processing application in order to process the artificial intelligence element; establishing a communication connection to an artificial intelligence processing module; sending the processing job to the artificial intelligence processing module; receiving results of the processing job from the artificial intelligence processing module; and completing the image processing task.

According to one aspect of the disclosure, there is provided a method of processing data from a tissue sample, as may be performed in a laboratory information system or other computer network environment, the method comprising:
loading from a patient record stored in a data repository a histological image of a section of a tissue sample into a convolutional neural network, the histological image including a two-dimensional array of pixels;
applying the convolutional neural network, CNN, to generate an output image with a two-dimensional array of pixels with a mapping to that of the histological image, the output image being generated by assigning one of a plurality of tissue classes to each pixel, wherein the plurality of tissue classes includes at least one class representing non-tumorous tissue and at least one class representing tumorous tissue;
determining, for each tissue class of clinical relevance, with reference to a stored protocol for that tissue class, which may for example be stored in a laboratory information system, whether any further tests should be performed on the tissue sample; and
creating and submitting an order for each further test that is to be performed.

In certain embodiments, once any further tests have been performed, their test results can be saved to the patient record.

With the proposed automated workflow, it is possible to eliminate the pathologist's intermediate examination of, for example, an initially provided H&E (hematoxylin and eosin) image (or other initial image, such as an unstained image), since, at the time of the pathologist's first review, not only will the H&E image be available to view, but also results, in particular further images from additional tests carried out in response to the automated CNN-based image processing of the initial image.

The proposed automation of the workflow prior to first review by a pathologist thus shortens the time between biopsy and diagnosis, since the computer-automated CNN-processing of the digital H&E slide, or other initially scanned histological image, can be performed immediately after obtaining the digital slide. Significant wait time between the initial digital image being obtained, e.g. an image of the H&E-stained slide, by the digital scanner and any necessary additional tests being ordered can therefore be eliminated. In a traditional workflow, this wait time can be significant, not only since it requires a pathologist to be available to review the H&E slide, but also since it is common to have a workflow in which the pathologist's review of the H&E slide is linked to a patient appointment, so the review waits for this appointment to take place and the additional tests are only ordered during or immediately after this patient appointment.

The histological image may include one or more further two-dimensional array of pixels and thus include a plurality of two-dimensional arrays of pixels, as may be the case, e.g. one for each of a plurality of stains, or one for each of a different depth in the sample (a so-called z-stack) obtained by stepping the focal plane of the microscope through a transparent or semi-transparent sample of finite depth. The output image generated by the CNN will also comprise one or more two-dimensional array of pixels, wherein there is a defined mapping between the (input) histological image(s) and the output image(s), where this may be a one-to-one mapping, a many-to-one mapping or a one-to-many mapping. The histological image processed by the CNN may be an H&E image. In the specific example of the histological image to which the CNN is applied being an H&E image, the CNN-processing applies a tumor finding and classification algorithm to the H&E slide to identify tumorous tissue and the tissue type. If the H&E slide contains tumorous tissue that is of a tissue type that requires additional testing before a reliable diagnosis is possible, the additional tests are ordered automatically by an order-placing algorithm which takes as input the output of the CNN. The digital scanning of the H&E slide, the CNN processing and the subsequent ordering of additional tests, and the further digital scanning of any further slides associated with the additional tests, can be coordinated by a single computer program in a single automated workflow, so can be integrated in a laboratory information system and a wider clinical network such as a hospital network or another kind of computer network, such as in a research laboratory.

Providing that at least one pixel of a clinically relevant tissue class is present in the output image from the CNN, a filter may be applied to screen pixels of that tissue class to determine whether they are present with a significant abundance, wherein, whether an order for any further tests is created for that tissue class is conditional on determining that there is a significant abundance of pixels of that tissue class.

The automatically ordered additional test may relate to obtaining one or more further histological images, e.g. from a further tissue section of the same sample that has been labeled with a different stain or marker, or may be any other kind of test that is envisaged by the protocol as being relevant for tumors of a particular class. The marker may be selected from the group: ER (estrogen receptor), PR (progesterone receptor) and HER2 (human epidermal growth factor receptor 2). The histological image and the further histological image may be displayed on a display.

In some embodiments, creating and submitting each order may be made further conditional on checking whether an authorization is needed for that order and, if not already provided for, issuing a request to a user to seek such an authorization.

The stored protocols associated with respective tissue classes may be organized in a database. Determining whether any further tests are to be performed can then be carried out by submitting a database query containing at least one the tissue classes identified in the sample by the CNN. Determining whether any further tests are to be performed may also be made conditional on a reference to the patient record to check that results of such a further test are not already available.

The workflow may be integrated with the original image acquisition in a slide scanner. For example, the CNN may be applied directly after the image acquisition by the slide scanner. The slide scanner may automatically save acquired images to a virtual slide library, for example a database located in the hospital or laboratory network, and newly acquired images of certain types may then trigger the automated test ordering method to be performed.

In our current implementation, in each successive convolution stage, as the dimensions decrease, the depth increases, so that the convolution layers are of ever increasing depth as well as ever decreasing dimensions, and in each successive transpose convolution stage, as the dimensions increase, the depth decreases, so that the deconvolution layers are of ever decreasing depth as well as ever increasing dimensions. The final convolution layer then has a maximum depth as well as minimum dimensions. Instead of the approach of successive depth increases and decreases through respectively the convolution and deconvolution stages, an alternative would be to design a neural network in which every layer except the input layer and the output layer has the same depth.

The method may further comprise: displaying on a display the histological image or set thereof with the probability map, e.g. overlaid thereon or alongside each other. The probability map can be used to determine which areas should be scored by whatever immunohistochemistry (IHC) scoring algorithms are to be used. The probability map can also be used to generate a set of contours around tumor cells which can be presented in the display, e.g. to allow a pathologist to evaluate the results generated by the CNN.

The CNN may be configured to receive as input the histological image in patches, in which case the CNN will output correspondingly sized patches. The output image patches would then be subsequently assembled into a probability map covering the histological image. After the assembling step, the probability map may be stored into the record in the data repository, so that the probability map is linked to the histological image or set thereof.

In certain embodiments, the convolutional neural network has one or more skip connections. Each skip connection takes intermediate results from at least one of the convolution layers of larger dimensions than the final convolution layer and subjects those results to as many transpose convolutions as needed, which may be none, one or more than one, to obtain at least one further recovered layer matched in size to the input image patch. These are then combined with the above-mentioned recovered layer prior to said step of assigning a tissue class to each pixel. A further processing step combines the recovered layer with each of the further recovered layers in order to recompute the probabilities, thereby taking account of the results obtained from the skip connections.

In certain embodiments, a softmax operation is used to generate the probabilities.

The image patches extracted from the histological image(s) may cover the whole area of the image(s). The patches may be non-overlapping image tiles or image tiles that overlap at their margins to aid stitching of the probability map. While each image patch should have a fixed number of pixels in width and height to be matched to the CNN, since the CNN will be designed to accept only a fixed size of pixel array, this does not mean that each image patch must correspond to the same physical area on the histological image, because pixels in the histological image may be combined into a lower resolution patch covering a larger area, e.g. each 2×2 array of neighboring pixels may be combined into one ‘super’-pixel to form a patch with four times the physical area of a patch extracted at the native resolution of the histological image.

The method can be performed for prediction once the CNN has been trained. The purpose of the training is to assign suitable weight values for the inter-layer connections. For training, the records that are used will include ground truth data which assigns each pixel in the histological image or set thereof to one of the tissue classes. The ground truth data will be based on use of an expert clinician to annotate a sufficiently large number of images. Training is carried out by iteratively applying the CNN, where each iteration involves adjusting the weight values based on comparing the ground truth data with the output image patches. In our current implementation, the weights are adjusted during training by gradient descent.

There are various options for setting the tissue classes, but most if not all embodiments will have in common that a distinction will be made in the classes between non-tumorous and tumorous tissue. The non-tumorous tissue classes may include one, two or more classes. There may also be a class representing areas where no tissue is identified, i.e. blank areas on the slide, which may in particular be useful for tissue microarray samples. The tumorous tissue classes may also include one, two or more classes. For example, in our current implementation we have three tissue classes, one for non-tumorous tissue and two for tumorous tissue, wherein the two tumorous tissue classes are for invasive tumors and in situ tumors.

In some embodiments the CNN is applied to one histological image at a time. In other embodiments the CNN may be applied to a composite histological image formed by combining a set of histological images taken from differently stained, adjacent sections of a region of tissue. In still further embodiments, the CNN may be applied in parallel to each of the images of a set of images taken from differently stained, adjacent sections of a region of tissue.

With the results from the CNN, the method may be extended to include a scoring process based on the pixel classification and the tumors that are defined from that classification with reference to the probability map. For example, the method may further comprise: defining areas in the histological image that correspond to tumors according to the probability map; scoring each tumor according to a scoring algorithm to assign a score to each tumor; and storing the scores into the record in the data repository. The scoring thus takes place on the histological image, but is confined to those areas identified by the probability map as containing tumorous tissue.

The results may be displayed on a display to a clinician. Namely, a histological image can be displayed with its associated probability map, e.g. overlaid thereon or alongside each other. The tumor scores may also be displayed in some convenient manner, e.g. with text labels on or pointing to the tumors, or alongside the image.

According to a further aspect of the disclosure, there is provided a computer program product bearing machine readable instructions for performing the above-described method.

A still further aspect of the invention relates to a computer network system, such as in a hospital, clinic, laboratory or research facility, for processing data from a tissue sample, the system comprising:
a data repository operable to store patient records containing histological images of sections of tissue samples, the histological image including a two-dimensional array of pixels;
a processing module loaded with a computer program configured to receive histological images from the patient records and apply a convolutional neural network thereto so as to generate an output image with a two-dimensional array of pixels with a mapping to that of the histological image, the output image being generated by assigning one of a plurality of tissue classes to each pixel, wherein the plurality of tissue classes includes at least one class representing non-tumorous tissue and at least one class representing tumorous tissue;
a test ordering module loaded with a computer program configured to:
determine for at least one of the tissue classes with reference to a protocol for that tissue class stored in the computer network system whether any further tests should be performed on the tissue sample;
create and submit an order within the computer network system for each further test that is to be performed; and
save test results from each further test to the patient record.

In certain embodiments, the processing module comprises:
an input operable to receive a histological image or set thereof from a record stored in a data repository;
a pre-processing module configured to extract image patches from the histological image or set thereof, the image patches being area portions of the histological image or set thereof having a size defined by numbers of pixels in width and height; and
a convolutional neural network with a set of weights and a plurality of channels, each channel corresponding to one of a plurality of tissue classes to be identified, wherein at least one of the tissue classes represents non-tumorous tissue and at least one of the tissue classes represents tumorous tissue, the convolutional neural network being operable to:
   receive as input each image patch as an input image patch;
   perform multi-stage convolution to generate convolution layers of ever decreasing dimensions up to and including a final convolution layer of minimum dimensions, followed by multi-stage transpose convolution to reverse the convolutions by generating deconvolution layers of ever increasing dimensions until a layer is recovered matched in size to the input image patch, each pixel in the recovered layer containing a probability of belonging to each of the tissue classes; and
   assign a tissue class to each pixel of the recovered layer based on said probabilities to arrive at an output image patch.

The system may further comprise: a post-processing module configured to assemble the output image patches into a probability map for the histological image or set thereof. Moreover, the system may further comprise: an output operable to store the probability map into the record in the data repository, so that the probability map is linked to the histological image or set thereof. The system may still further comprise: a display and a display output operable to transmit the histological image or set thereof and the probability map to the display such that the histological image is displayed with the probability map, e.g. overlaid thereon or alongside the probability map.

The system may additionally comprise an image acquisition apparatus, such as a microscope, operable to acquire histological images or sets thereof and to store them to records in the data repository.

It will be understood that in at least some embodiments the histological image(s) are digital representations of a two-dimensional image taken of a sectioned tissue sample by a microscope, in particular a light microscope, which may be a conventional optical microscope, a confocal microscope or any other kind of microscope suitable for obtaining histological images of unstained or stained tissue samples. In the case of a set of histological images, these may be of a succession of microscope images taken of adjacent sections (i.e. slices) of a region of tissue, wherein each section may be differently stained.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is an overview block diagram of a system according to the disclosure.

FIG. 2 shows in more detail some of the system elements of FIG. 1, in particular the AI processing module and its configuration module.

FIG. 3 shows in more detail some of the system elements of FIG. 1, in particular the digital pathology application module.

FIG. 4 shows more details of the inputs and outputs of the digital pathology application module of FIG. 3.

FIG. 5A is a schematic drawing of a neural network architecture used in one embodiment of the invention.

FIG. 5B shows how within the neural network architecture of FIG. 5A global and local feature maps are combined to generate a feature map that predicts an individual class for each pixel in an input image patch.

FIG. 6A is a drawing showing a raw digital pathology image that in operation is a color image.

FIG. 6B is a drawing showing the CNN prediction of FIG. 6A that in operation is a color image. The CNN prediction image illustrates non-tumor area (green), invasive tumor area (red), and non-invasive tumor (blue).

FIG. 7A is a drawing showing an example of the input RGB image patch that in operation is a color image. The image patch shows the pathologist's manual outlining of invasive tumors (red) and additionally shows overlays of the neural network's predictions (pink and yellow).

FIG. 7B is a drawing showing the final output tumor probability heat map that in operation is a color image. The heat map shows overlays of the neural network's predictions (in reddish-brown and blue respectively).

FIG. 8 is a flow diagram showing the steps involved in training the CNN.

FIG. 9 is a flow diagram showing the steps involved in prediction using the CNN.

FIG. 10 is a flow diagram of a method according to an embodiment of the present disclosure.

FIG. 11 is a block diagram of a TPU which may be used for performing the computations involved in implementing the neural network architecture of FIGS. 5A and 5B.

FIG. 12 shows an example computer network which can be used in conjunction with embodiments of the invention.

FIG. 13 is a block diagram of a computing apparatus that may be used for example as the host computer for the TPU of FIG. 11.

FIG. 14A is a block diagram illustrating an example processor enabled device 550 that may be used in connection with various embodiments described herein;

FIG. 14B is a block diagram illustrating an example line scan camera having a single linear array.

FIG. 14C is a block diagram illustrating an example line scan camera having three linear arrays; and

FIG. 14D is a block diagram illustrating an example line scan camera having a plurality of linear arrays.

### DETAILED DESCRIPTION

In the following detailed description, for purposes of explanation and not limitation, specific details are set forth in order to provide a better understanding of the present disclosure. It will be apparent to one skilled in the art that the present disclosure may be practiced in other embodiments that depart from these specific details.

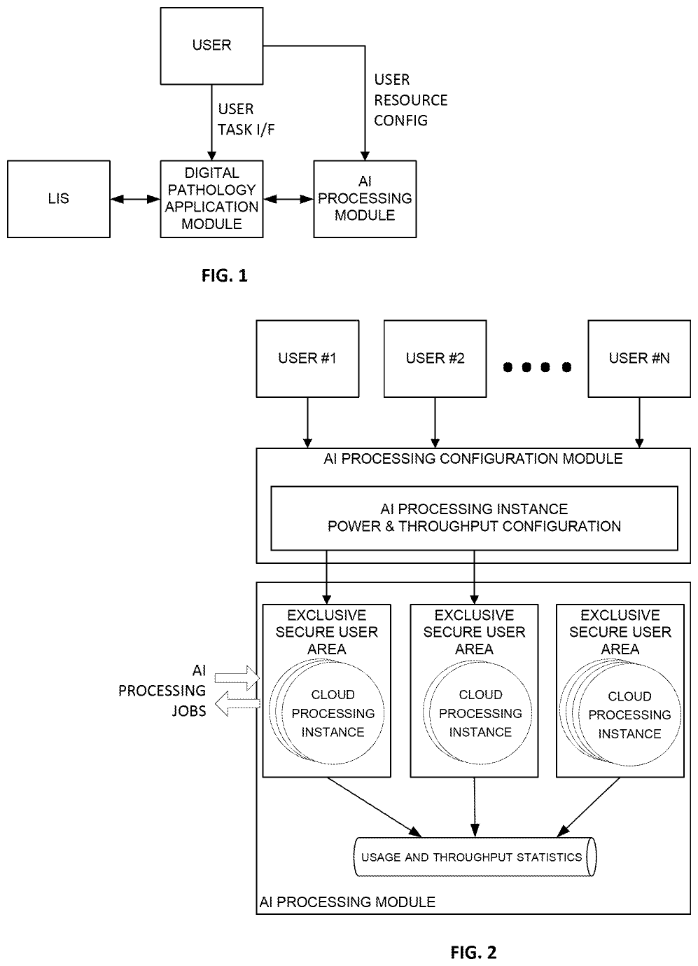

FIG. 1 is an overview block diagram of a system according to the disclosure. The system facilitates and orchestrates the distribution of artificial intelligence (AI) processing functionality to processing instances. The system is able to manage the delegation of AI processing jobs to the processing instances and the receipt of results data from these processing jobs, including amalgamation of the results data into a coherent and complete result set. The system supports a user configuring the throughput and processing power characteristics of the processing instances within their own cloud processing area. The system provides mechanisms that may be used to initiate the execution of a digital pathology application, including user-initiated mechanisms and mechanisms triggered via an external event, such as another application.

The system comprises a laboratory information system (LIS) which may be part of a larger clinical network environment, such as a hospital information system (HIS) or picture archiving and communication system (PACS). In the LIS, the WSIs will be retained in a database as virtual slides, typically the database is a patient information database containing the electronic medical records of individual patients. The WSIs will be taken from stained tissue samples mounted on slides, the slides bearing printed barcode labels by which the WSIs are tagged with suitable metadata, since the microscopes acquiring the WSIs are equipped with barcode readers. From a hardware perspective, the LIS will be a conventional computer network, such as a local area network (LAN) with wired and wireless connections as desired.

The system further comprises a digital pathology application module, which is configured to host one or more digital pathology applications, which in the context of the present application includes a digital pathology application which relies on artificial intelligence (AI) processing, such as using a convolutional neural network (CNN). The digital pathology application module has a user interface, through which the user may allocate tasks to a digital pathology application running on the digital pathology application module.

The system further comprises an AI processing module, an example tumor-finding CNN functionality of which is described in detail further below. The AI processing module is in operative connection to the digital pathology application module, so that AI jobs can be sent from the digital pathology application module to the AI processing module where they are processed, and then returned to the digital pathology application module with the AI processing results. The AI processing module has a user interface for configuring resources, through which a user can reserve and configure AI processing capacity, for example as specified by processing power or throughput configuration, and as described further below with reference to service models.

FIG. 2 shows in more detail some of the system elements of FIG. 1, in particular the AI processing module. Intermediate between users and the AI processing module, an AI processing configuration module is provided which allows a user to reserve and configure AI processing capacity, for example as specified by processing power or throughput configuration, and as described further below with reference to service models. A user may interact with the configuration module to reserve and manage a user area within the AI processing module, where the user area is provided with appropriate security and may be exclusive to the user. Processing power may be reserved exclusively for a user or reserved in a pooled arrangement with other users. The system will facilitate each user or user group configuring the execution characteristics of their own image processing area on the cloud. Configuration options available to the user will include (and may not be limited to):
the processing power of each processing instance;
the quantity of processing instances that may be run or invoked simultaneously;
the maximum number of processing instances that may run at any given time; and
the maximum execution time per period (e.g. 5 hours of processing time per hour, 100 hours of processing time per day, etc.).

Three example user areas are schematically illustrated, each with different numbers of processing instances reserved. Usage and throughput of the user areas generates statistical data which may be collected by a monitoring unit of the AI processing module. Usage statistics will be gathered concerning the processing characteristics of the user/user-group's exclusive processing area. Such statistics may include data about when and for how long each processing instance is active and processing. It is envisaged that the usage statistics do not include any patient data, in particular any data that is attributable to an individual patient, such as: patient image data, execution initialization or metadata or case-identifiable data. The monitoring unit may compile and output these statistics, for example on a periodic basis, such as monthly.

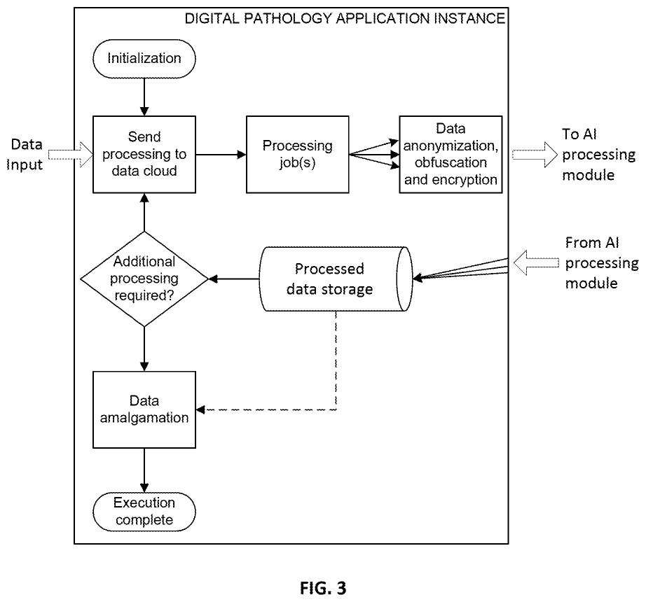

FIG. 3 shows in more detail some of the system elements of FIG. 1, in particular the digital pathology application instance as may run on the corresponding module. An example internal workflow of an application is shown. An instance of the digital pathology application may be initiated via several mechanisms, e.g.:
direct user interaction with the application module via a UI
an internal trigger from an event in the application module
user interaction with the LIS
an internal event in the LIS

Patient data, such as histological image data may be sourced as follows. Upon application initialization, data (both image data and any required metadata or other processing data) will be sourced according to the conditions by which the application instance was triggered. For example, an analysis trigger event from the digital pathology application module may necessitate data being sourced internally, whereas an analysis trigger event from the LIS may necessitate data being sourced from the LIS.

Application instances need not be provided with comprehensive patient data. Usually, image analysis application instances require only the image data itself and occasionally application configuration data. The application may assess the characteristics of the image data so as to identify opportunities to parallelize the image processing required, which may include AI processing jobs. Possible parallel processing could be based on processing different whole histological images, or processing different subsets of a histological image in parallel, where the subsets may be based on image tiles, or channels in the case of a multichannel histological image. Specific protocols such as for Fluorescence in situ hybridization (FISH) may also provide parallelization opportunities, where for FISH the finding of nuclear areas and of signal could be processed in parallel. In preparation of an AI processing job for allocation to the AI processing module, the application may select only the data that is needed by the AI processing instance to complete the processing of that job. Data that is unnecessary will be omitted from the processing job. In that way confidential patient data may be omitted from the data set collected for the AI processing job. If the data set collected for the AI processing job does include data which is patient confidential, the AI processing job may be further modified to anonymize, obfuscate and/or encrypt such data before sending the job to the AI processing instance. More than one iteration of cloud processing may be required by an algorithm since the number of parallelizable jobs that can be executed by a processing instance may exceed the number of processing instances available. For this reason, the algorithm will likely orchestrate many calls to process instances as process instances start processing jobs, complete these jobs and become available for another job.

When processing a job, as each AI processing instance returns its processing data to the application module, this data will be amalgamated by a data amalgamation unit within the digital pathology application. This will facilitate a cohesive and overall result set to be constructed in preparation for this to be returned to the calling function, which may be the digital pathology application module, the LIS or some other module within the system.

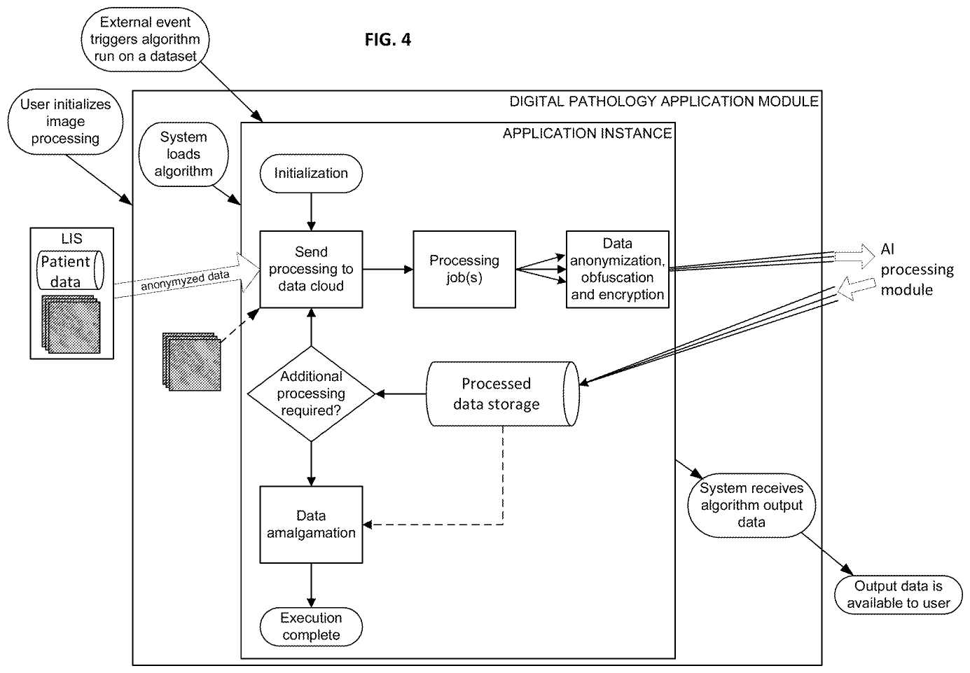

FIG. 4 shows more details of the inputs and outputs of the digital pathology application module of FIG. 3. The application module may be configured by a user to initialize image processing. An application instance can be loaded in response to a request from a user, or other external or internal request source. Histological image data from virtual slides is shown schematically stored in the LIS and also stored within the application module. The transfer of image data from the LIS to the application module may be anonymized and/or encrypted. As described above, users will have the facility to specify the number and processing power of AI processing instances that they require be made available. AI processing instances, upon initialization via newly arriving AI processing instance data from the digital pathology application instance, will decrypt the job data. The AI processing instance will then process the data and return it to the calling function. The AI processing instance is configured with a data retention policy which promptly and permanently deletes image data and any other potentially patient-confidential data contained in the AI jobs it receives, as well as its output data sent back to the calling function, as soon as practically possible after the AI job has been closed. Each processing instance will however compile data regarding when it was initialized and how long it spent processing, and this data will be collated within the AI processing module and may provide a statistical summary of usage both for the user and for the AI processing module administrator. The application instance may then output the results of any AI processing job, or wider task which included AI processing either internally or externally, e.g. to a user or to the LIS, as dictated by its application process flow.

In an extension of the above, the application instance may be further encapsulated in an agnostic wrapper which provides a container to standardize the data input and output and therefore decouple the application instance from being tied to particular data input or output formats or standards. The agnostic wrapper provides an interface between the application instance and external elements so as to handle external functions such as initialization, accepting processing data, interaction with external AI processing instances and also the return of AI processing data. In harnessing the input and output facilities specified in the interface, the agnostic wrapper facilitates diverse initialization scenarios, while standardizing data input and output structure. The system also supports the setup and configuration of externally-hosted AI processing instances, which may exist on a variety of different cloud architectures. The agnostic wrapper is capable of initialization, is capable of functioning and processing within the system and is capable of returning data to the system; all within a structure provided via the defined algorithm interfaces within the system. The agnostic wrapper described here provides example usages of defined interfaces to achieve an image processing task. Initialization is made having regard to metadata about the environment in which the agnostic wrapper and the application instance contained therein is running, guidance of the processing instances to be used if parallel processing is to be used as well as other metadata. Data may also be included regarding other processing tasks that may need to be run based on output data from the application instance, or other applications that may be called by the application instance contained in the agnostic wrapper, e.g. based on output from the application instance.

An interface function, “Provide Input Data to Application Instance”, is provided to allow necessary input data to be routed to the application instance. The system facilitates the routing of output data from a completed application instance to the input of a new application instance. This routing, in conjunction with the facility to specify secondary, tertiary etc. application instances during initialization, can be used to achieve “daisy-chaining” of application instances. The defined interface also specifies a function that can be used for the export of processing data to AI processing instances and function that can be used to receive analysis data from AI processing instances. The defined interface specifies a “Receive Result Data” function to which data can be sent as AI processing completes.

Tests and decision points within the agnostic wrapper, but outside the application instance, ascertain whether and to what extent local processing within the application instance is required and whether and to what extent processing should be outsourced, e.g. to the AI processing module. The tests and decision points may also provide for the identification and outsourcing of parallelizable AI processing jobs. The algorithm, having assessed image characteristics of the input image data, is capable of identifying opportunities to parallelise the AI elements of the image processing through generation of AI processing jobs to be sent to the AI processing module for execution. In preparation for the agnostic wrapper (including the contained application instance) to send each processing job to a processing instance, the agnostic wrapper may select the data necessary for the AI processing instance to complete the processing of that job, i.e. not include any data that is not necessary for executing the processing job, so that for example patient data and a macro image may be omitted. Additionally, data destined for a AI processing instance may be encrypted before sending to the AI processing instance.

The agnostic wrapper provides for the possibility of Iterative execution of processing within the application instance. A decision point, “Additional analysis required” exists to allow iterations of local processing and/or external processing. Upon the completion of the required processing, data is gathered from “Analysis data accumulator” in preparation to export. Initialization may include information about a user area that a user may require to be used when sending pit AI processing jobs to AI processing instances. In this way the user who instructs the agnostic wrapper to cause the application instance to run a task can also designate to which AI processing modules or instances that the application instance may send AI processing jobs.

It will be understood that the system as described with reference to FIGS. 1 to 4 may be implemented in part or entirely in a cloud-computing environment as described in more detail further below. Moreover, it will be understood that any of the above-mentioned modules and also the LIS may be a network node, or be associated with a network node, in a distributed system.

AI Processing Module Functionality

One example of an AI processing function that may be provided by and ran on the AI processing module is a convolutional neural network (CNN). The CNN may be designed for tumor finding in a digital pathology histological image, e.g. to classify each image pixel into either a non-tumor class or one of a plurality of tumor classes. In the following by way of example we refer to breast cancer tumors. The neural network in our example implementation is similar in design to the VGG-16 architecture available at: <http://www.robots.ox.ac.uk/˜vgg/research/very_deep/> and described in Simonyan and Zisserman 2014, the full contents of which are incorporated herein by reference. We describe operation of the system in the context of a CNN tumor finding application which detects and outlines invasive and in situ breast cancer cell nuclei automatically. The method is applied to a single input image, such as a WSI, or a set of input images, such as a set of WSIs. Each input image is a digitized, histological image, such as a WSI. In the case of a set of input images, these may be differently stained images of adjacent tissue sections. We use the term stain broadly to include staining with biomarkers as well as staining with conventional contrast-enhancing stains. Since CNN-based automatic outlining of tumors is much faster than manual outlining, it enables an entire image to be processed, rather than only manually annotating selected extracted tiles from the image. The automatic tumor outlining should thus enable pathologists to compute a positivity (or negativity) percentage over all the tumor cells in the image, which should result in more accurate and reproducible results.

The input image is a pathology image stained with any one of several conventional stains as discussed in more detail elsewhere in this document. For the CNN, image patches are extracted of certain pixel dimensions, e.g. 128×128, 256×256, 512×512 or 1024×1024 pixels. It will be understood that the image patches can be of arbitrary size and need not be square, but that the number of pixels in the rows and columns of a patch conform to 2n, where n is a positive integer, since such numbers will generally be more amenable for direct digital processing by a suitable single CPU (central processing unit), GPU (graphics processing unit) or TPU (tensor processing unit), or arrays thereof.

We note that ‘patch’ is a term of art used to refer to an image portion taken from a WSI, typically with a square or rectangular shape. In this respect we note that a WSI may contain a billion or more pixels (gigapixel image), so image processing will typically be applied to patches which are of a manageable size (e.g. ca. 500×500 pixels) for processing by a CNN. The WSI will thus be processed on the basis of splitting it into patches, analyzing the patches with the CNN, then reassembling the output (image) patches into a probability map of the same size as the WSI. The probability map can then be overlaid, e.g. semi-transparently, on the WSI, or part thereof, so that both the pathology image and the probability map can be viewed together. In that sense the probability map is used as an overlay image on the pathology image. The patches analyzed by the CNN may be of all the same magnification, or may have a mixture of different magnifications, e.g. 5×, 20×, 50× etc. and so correspond to different sized physical areas of the sample tissue. By different magnifications, these may correspond to the physical magnifications with which the WSI was acquired, or effective magnifications obtained from digitally downscaling a higher magnification (i.e. higher resolution) physical image.

A recent trend in pathology is that convolutional neural network (CNN) methods have become of increasing research interest. It is becoming increasingly reported that CNN methods are performing as well as, or even better than, pathologists in identifying and diagnosing tumors from histological images.

Wang et al 2016 describes a CNN approach to detect metastasis of breast cancer to the lymph nodes.

US2015213302A1 describes how cellular mitosis is detected in a region of cancerous tissue. After training a CNN, classification is carried out based on an automated nuclei detection system which performs a mitotic count, which is then used to grade the tumor.

Hou et al 2016 processes brain and lung cancer images. Image patches from WSIs are used to make patch-level predictions given by patch-level CNNs.

Liu et al 2017 processes image patches extracted from a gigapixel breast cancer histological image with a CNN to detect and localize tumors by assigning a tumor probability to every pixel in the image.

Bejnordi et al 2017 applies two stacked CNNs to classify tumors in image patches extracted from WSIs of breast tissue stained with a hematoxylin and eosin (H&E) stain. The performance is shown to be good for object detection and segmentation in these pathology images. We further note that Bejnordi et al also provides an overview of other CNN-based tumor classification methods applied to breast cancer samples (see references 10-13).

Esteva et al 2017 applies a deep CNN to analyze skin lesions and classify the lesions according to a tree-structured taxonomy into various malignant types, non-malignant types and non-neoplastic types including the malignant types acrolentiginous melanoma, amelanotic melanoma and lentigo melanoma and the non-malignant types blue nevus, halo nevus and mongolian spot. An image of a skin lesion (for example, melanoma) is sequentially warped into a probability distribution over clinical classes to perform the classification.

Mobadersany et al 2017 disclose a computational method based on a survival CNN to predict the overall survival of patients diagnosed with brain tumors. Pathology image data from tissue biopsies (histological image data) is fed into the model as well as patient-specific genomic biomarkers to predict patient outcomes. This method uses adaptive feedback to simultaneously learn the visual patterns and molecular biomarkers associated with patient outcomes.

In the following, we describe a CNN-based, computer-automated tumor finding method which detects and outlines invasive and in situ breast cancer cell nuclei automatically. The method is applied to a single input image, such as a WSI, or a set of input images, such as a set of WSIs. Each input image is a digitized, histological image, such as a WSI. In the case of a set of input images, these may be differently stained images of adjacent tissue sections. We use the term stain broadly to include staining with biomarkers as well as staining with conventional contrast-enhancing stains.

Since computer-automated outlining of tumors is much faster than manual outlining, it enables an entire image to be processed, rather than only manually-annotating selected extracted tiles from the image. The proposed automatic tumor outlining should thus enable pathologists to compute a positivity (or negativity) percentage over all the tumor cells in the image, which should result in more accurate and reproducible results.

The proposed computer-automated method for tumor finding, outlining and classifying uses a convolutional neural network (CNN) to find each nuclear pixel on the WSI and then to classify each such pixel into one of a non-tumor class and one of a plurality of tumor classes, in our current implementation breast tumor classes.

The neural network in our implementation is similar in design to the VGG-16 architecture available at: http://www.robots.ox.ac.uk/˜vgg/research/very_deep/ and described in Simonyan and Zisserman 2014, the full contents of which are incorporated herein by reference.

The input image is a pathology image stained with any one of several conventional stains as discussed in more detail elsewhere in this document. For the CNN, image patches are extracted of certain pixel dimensions, e.g. 128×128, 256×256, 512×512 or 1024×1024 pixels. It will be understood that the image patches can be of arbitrary size and need not be square, but that the number of pixels in the rows and columns of a patch conform to 2n, where n is a positive integer, since such numbers will generally be more amenable for direct digital processing by a suitable single CPU (central processing unit), GPU (graphics processing unit) or TPU (tensor processing unit), or arrays thereof.

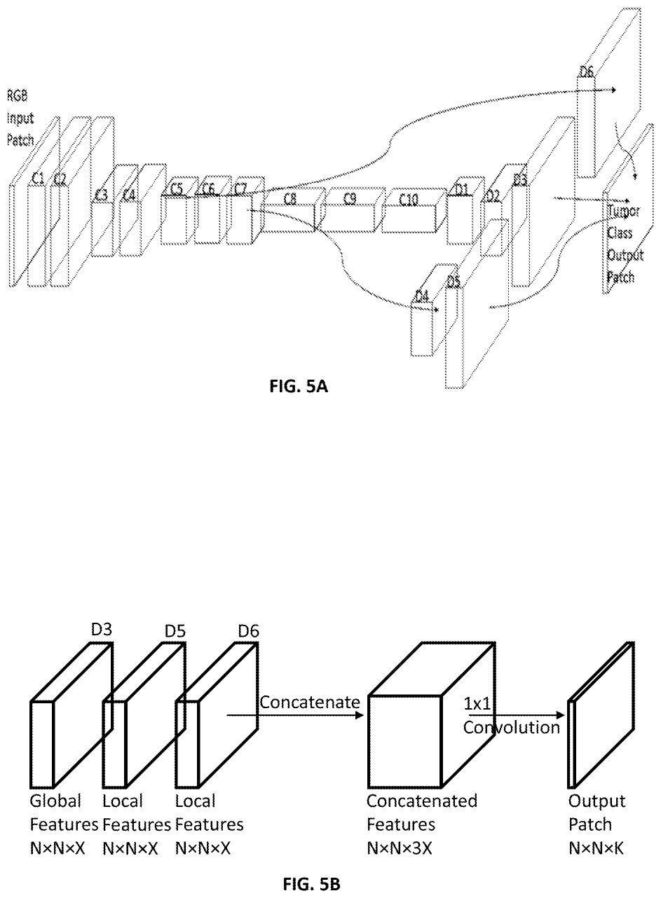

FIG. 5A is a schematic drawing of our neural network architecture. Layers C1, C2 . . . C10 are convolutional layers. Layers D1, D2, D3, D4, D5 and D6 are transpose convolution (i.e. deconvolutional) layers. The lines interconnecting certain layers indicate skip connections between convolutional, C, layers and deconvolutional, D, layers. The skip connections allow local features from larger dimension, shallower depth layers (where “larger” and “shallow” mean a convolutional layer of lower index) to be combined with the global features from the last (i.e. smallest, deepest) convolutional layer. These skip connections provide for more accurate outlines. Maxpool layers, each of which is used to reduce the width and height of the patch by a factor of 2, are present after layers C2, C4 and C7, but are not directly shown in the schematic, although they are shown by implication through the consequential reducing size of the patch. In some implementations of our neural network the maxpool layers are replaced with 1×1 convolutions resulting in a fully convolutional network.

The convolutional part of the neural network has the following layers in sequence: input layer (RGB input image patch); two convolutional layers, C1, C2; a first maxpool layer (not shown); two convolutional layers C3, C4; a second maxpool layer (not shown); three convolutional layers, C5, C6, C7, and a third maxpool layer (not shown). The output from the second and third maxpool layers is connected directly to deconvolutional layers using skip connections in addition to the normal connections to layers C5 and C8 respectively.

The final convolutional layer, C10, the output from the second maxpool layer (i.e. the one after layer C4) and the output from the third maxpool layer (i.e. the one after layer C7), are then each connected to separate sequences of “deconvolution layers” which upscale them back to the same size as the input (image) patch, i.e. convert the convolutional feature map to a feature map which has the same width and height as the input image patch and a number of channels (i.e. number of feature maps) equal to the number of tissue classes to be detected, i.e. a non-tumorous type and one or more tumor types. For the second maxpool layer, we see a direct link to the layer D6 since only one stage of deconvolution is needed. For the third maxpool layer, two stages of deconvolution are needed, via intermediate deconvolution layer D4, to reach layer D5. For the deepest convolutional layer C10, three stages of deconvolution are needed, via D1 and D2 to layer D3. The result is three arrays D3, D5, D6 of equal size to the input patch.

A simplified, albeit probably less-well performing, version of what is illustrated in FIG. 5A could omit the skip connections, in which case layers D4, D5 and D6 would not be present and the output patch would be computed solely from layer D3.

FIG. 5B shows in more detail how the final steps in the neural network architecture of FIG. 5A are carried out. Namely, global feature map layer D3 and local feature map layers D5, D6 are combined to generate a feature map that predicts an individual class for each pixel of the input image patch. Specifically, FIG. 5B shows how the final three transpose convolution layers D3, D5, D6 are processed to the tumor class output patch.

We now discuss how the above-described approach differs from a known CNN used currently in digital pathology. This known CNN assigns one class selected from multiple available classes to each image patch. Examples of such type of CNN are in the papers by Wang et al 2016, Liu et al 2017, Cruz-Roa et al 2017 and Vandenberghe et al 2017. However, what we have just described is that, within a given image patch, one class selected from multiple available classes is assigned to each and every pixel. Therefore, instead of generating a single class label for each image patch, our neural network outputs a class label for each individual pixel of a given patch. Our output patch has a one-to-one pixel-to-pixel correspondence with the input patch such that each pixel in the output patch has assigned to it one of the multiple available classes (non-tumor, tumor 1, tumor 2, tumor 3 etc.).

In such known CNNs, to assign a single class to each patch, a series of convolutional layers is employed followed by one or several fully connected layers, followed by an output vector which has as many values as there are classes to detect. The predicted class is determined by the location of the maximum value in the output vector.

A trained CNN will take, as input, pixels from a digital slide image and return a vector of probabilities for each pixel (Goodfellow, Bengio, and Courville 2016). The vector is of length N where N is the number of classes the CNN has been trained to detect. For example, if a CNN has been trained to distinguish between three classes, invasive tumor, in situ tumor and non-tumor, the vector v will be of length 3. Each coordinate in the vector indicates the probability that the pixel belongs to a specific class. So v[0] may indicate the probability that the pixel belongs to the invasive tumor class, v[1] the probability it belongs to the in situ class and v[2] the probability it belongs to the non-tumor class. The class of each pixel is determined from the probability vector. A simple method of assigning a pixel to a class is to assign it to the class for which it has the highest probability.

To predict the class of individual pixels, our CNN uses a different architecture following the convolutional layers. Instead of a series of fully connected layers, we follow the convolutional layers with a series of transpose convolutional layers. The fully connected layers are removed from this architecture. Each transpose layer doubles the width and height of the feature maps while at the same time halving the number of channels. In this manner, the feature maps are upscaled back to the size of the input patch.

In addition, to improve the prediction, we use skip connections as described in Long et al 2015, the full contents of which is incorporated herein by reference.

The skip connections use shallower features to improve the coarse predictions made by upscaling from the final convolutional layer C10. The local features from the skip connections contained in layers D5 and D6 of FIG. 5A are concatenated with the features generated by upscaling the global features contained in layer D3 of FIG. 5A from the final convolutional layer. The global and local feature layers D3, D5 and D6 are then concatenated into a combined layer as shown in FIG. 5B.

From the concatenated layer of FIG. 5B (or alternatively directly from the final deconvolutional layer D3 in the case that skip connections are not used), the number of channels is reduced to match the number of classes by a 1×1 convolution of the combined layer. A softmax operation on this classification layer then converts the values in the combined layer into probabilities. The output patch layer has size N×N×K, where N is the width and height in pixels of the input patches and K is the number of classes that are being detected. Therefore, for any pixel P in the image patch there is an output vector V of size K. A unique class can then be assigned to each pixel P by the location of the maximum value in its corresponding vector V.

The CNN thus labels each pixel as non-cancerous or belonging to one or more of several different cancer (tumor) types. The cancer of particular interest is breast cancer, but the method is also applicable to histological images of other cancers, such as cancer of the bladder, colon, rectum, kidney, blood (leukemia), endometrium, lung, liver, skin, pancreas, prostate, brain, spine and thyroid.

Our specific neural network implementation is configured to operate on input images having certain fixed pixel dimensions. Therefore, as a preprocessing step, both for training and prediction, patches are extracted from the WSI which have the desired pixel dimensions, e.g. N×N×n pixels, where n=3 in the case that each physical location has three pixels associated with three primary colors—typically RGB, when the WSI is a color image acquired by a conventional visible light microscope. (As mentioned further below ‘n’ may be 3 times the number of composited WSIs in the case the two or more color WSIs are combined.) Moreover ‘n’ would have a value of one in the case of a single monochrome WSI. To make training faster the input patches are also centered and normalized at this stage.

Our preferred approach is to process the entire WSI, or at least the entire area of the WSI which contains tissue, so the patches in our case are tiles that cover at least the entire tissue area of the WSI. The tiles may be abutting without overlap, or have overlapping edge margin regions of for example 1, 2, 3, 4, 5, 6, 7, 8, 9 or 10 pixels wide so that the output patches of the CNN can be stitched together taking account of any discrepancies. Our approach can however, if desired, also be applied to a random sample of patches over the WSI which are of the same or different magnification, as in the prior art, or as might be carried out by a pathologist.

Our neural network is similar in design to the VGG-16 architecture of Simonyan and Zisserman 2014. It uses very small 3×3 kernels in all convolutional filters. Max pooling is performed with a small 2×2 window and stride of 2. In contrast to the VGG-16 architecture, which has a series of fully connected layers after the convolutional layers, we follow the convolution layers with a sequence of “deconvolutions” (more accurately transpose convolutions) to generate segmentation masks. This type of upsampling for semantic segmentation has previously been used for natural image processing by Long et al 2015, the full contents of which are incorporated herein by reference.

Each deconvolutional layer enlarges the input feature map by a factor of two in the width and height dimensions. This counteracts the shrinking effect of the maxpool layers and results in class feature maps of the same size as the input images. The output from each convolution and deconvolutional layer is transformed by a non-linear activation layer. At present, the non-linear activation layers use the rectifier function ReLU (x)=max (0, x)ReLU(x)=max(0,x). Different activation functions may be used, such as ReLU, leaky ReLU, eLU, etc. as desired.

The proposed method can be applied without modification to any desired number of tissue classes. The constraint is merely the availability of suitable training data which has been classified in the manner that it is desired to replicate in the neural network. Examples of further breast pathologies are invasive lobular carcinoma or invasive ductal carcinoma, i.e. the single invasive tumor class of the previous example can be replaced with multiple invasive tumor classes. The accuracy of the neural network is mostly dictated by the number of images available for each class, how similar the classes are, and how deep the neural network can be made before running into memory restrictions. In general, high numbers of images per class, deeper networks and dissimilar classes lead to higher network accuracy.

A softmax regression layer (i.e. multinomial logistic regression layer) is applied to each of the channel patches to convert the values in the feature map to probabilities.

After this final transformation by the softmax regression, a value at location location (x, y) in a channel C in the final feature map contains the probability, P(x, y), that the pixel at location location (x, y) in the input image patch belongs to the tumor type detected by channel C.

It will be appreciated that the number of convolution and deconvolution layers can be increased or decreased as desired and subject memory limitations of the hardware running the neural network.

We train the neural network using mini-batch gradient descent. The learning rate is decreased from an initial rate of 0.1 using exponential decay. We prevent neural network overfitting by using the “dropout” procedure described by Srivastava et al 2014 [2017], the full contents of which are incorporated herein by reference. Training the network may be done on a GPU, CPU or a FPGA using any one of several available deep learning frameworks. For our current implementation, we are using Google Tensorflow, but the same neural network could have been implemented in another deep learning framework such as Microsoft CNTK.

The neural network outputs probability maps of size N×N×K, where N is the width and height in pixels of the input patches and K is the number of classes that are being detected. These output patches are stitched back together into a probability map of size W×H×K, where W and H are the width and height of the original WSI before being split into patches.

The probability maps can then be collapsed to a W×H label image by recording the class index with maximum probability at each location (x, y) in the label image.

In its current implementation, our neural network assigns every pixel to one of three classes: non-tumor, invasive tumor and in situ tumor.

When multiple tumor classes are used, the output image can be post-processed into a simpler binary classification of non-tumor and tumor, i.e. the multiple tumor classes are combined. The binary classification may be used as an option when creating images from the base data, while the multi-class tumor classification is retained in the saved data.

While the above description of a particular implementation for our invention has concentrated on a specific approach using a CNN, it will be understood that our approach can be implemented in a wide variety of different types of convolutional neural network. In general, any neural network that uses convolution to detect increasingly complex features and subsequently uses transpose convolutions (“deconvolutions”) to upscale the feature maps back to the width and height of the input image should be suitable.

### Example 1

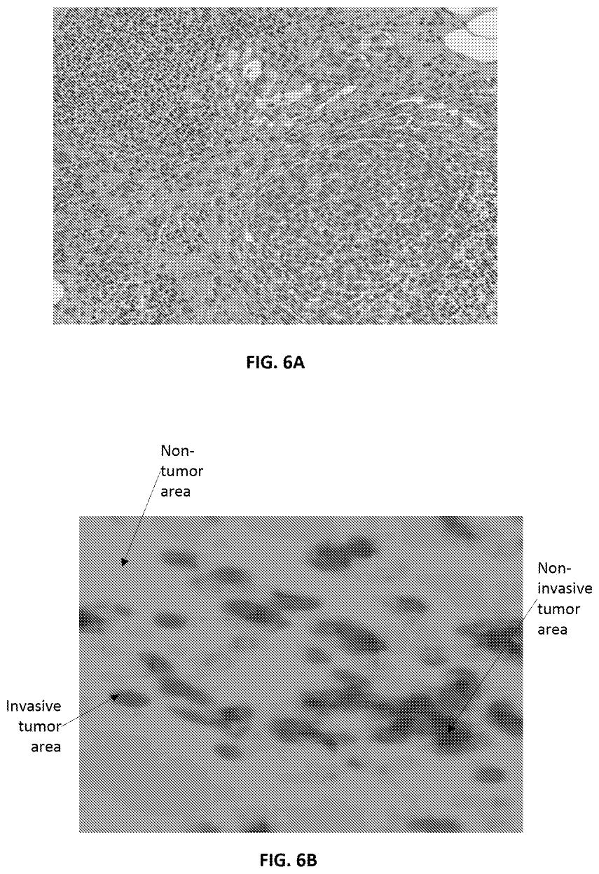

FIG. 6A is a color image in operation and shows the raw image. FIG. 6B is a color image in operation and shows the pixel-level predictions generated by the CNN.

FIG. 6A is a patch from an H&E-stained WSI in which the cluster of larger, dark purple cells in the bottom right quadrant is a tumor, while the smaller dark purple cells are lymphocytes.

FIG. 6B is a tumor probability heatmap generated by the CNN. It can be seen how the approach of pixel-level prediction produces areas with smooth perimeter outlines. For the heatmap, different (arbitrarily chosen) colors indicate different classes, namely green for non-tumor, red for a first tumor type and blue for a second tumor type.

### Example 2

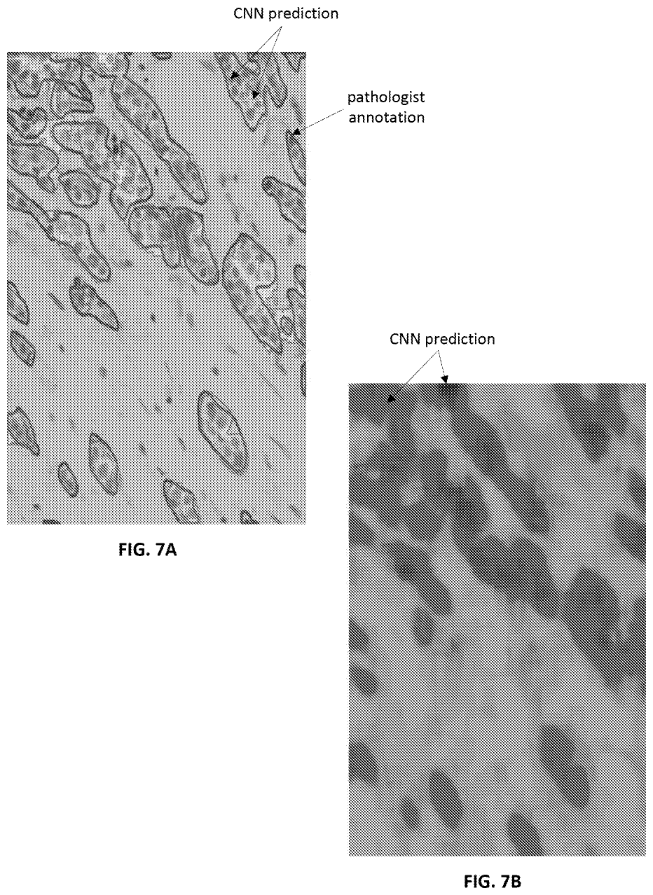

FIGS. 7A-7B are color images in operation and show an example of the input RGB image patch (FIG. 7A) and the final output tumor probability heat map (FIG. 7B).

FIG. 7A additionally shows the pathologist's manual outlining of invasive tumors (red outlines) along with overlays of the neural network's predictions (shaded pink and yellow areas).

FIG. 7B is a tumor probability heatmap generated by the CNN. For the heatmap, different (arbitrarily chosen) colors indicate different classes, namely green for non-tumor, reddish-brown for invasive tumor (correspondingly shown pink in FIG. 7A), and blue for in situ tumor (correspondingly shown yellow in FIG. 7A). Once again, it can be seen how the approach of pixel-level prediction produces areas with smooth perimeter outlines. Moreover, it can be seen how the CNN predictions are compatible with the pathologist's manual marking shown in FIG. 7A. In addition, the CNN provides a further distinction between invasive and non-invasive (in situ) tissue which was not carried out by the pathologist, and is inherently part of the multi-channel CNN design which can be programmed to and trained for classifying tissue into any number of different types as desired and clinically relevant.

Acquisition & Image Processing

The starting point of the method is that a tissue sample has been sectioned, i.e. sliced, and adjacent sections have been stained with different stains. The adjacent sections will have very similar tissue structure, since the sections are thin, but will not be identical, since they are of different layers.

For example, there could be 5 adjacent sections, each with a different stain, such as ER, PR, p53, HER2, H&E and Ki-67. A microscope image is then acquired of each section. Although the adjacent sections will have very similar tissue shapes, the stains will highlight different features, e.g. nucleus, cytoplasm, all features by general contrast enhancement etc.

The different images are then aligned, warped or otherwise pre-processed to map the coordinates of any given feature on one image to the same feature on the other images. The mapping will take care of any differences between the images caused by factors such as slightly different magnifications, orientation differences owing to differences in slide alignment in the microscope or in mounting the tissue slice on the slide, and so forth.

It is noted that with a coordinate mapping between different WSIs of a set comprising differently stained adjacent sections, the WSIs can be merged into a single composite WSI from which composite patches may be extracted for processing by the CNN, where such composite patches would have dimensions N×N×3 m, where ‘m’ is the number of composited WSIs forming the set.

Some standard processing of the images is then carried out. These image processing steps may be carried out on the WSI level or at the level of individual image patches. The images may be converted from color to grayscale if the CNN is configured to operate on monochrome rather than color images. The images may be modified by applying a contrast enhancement filter. Some segmentation may then be performed to identify common tissue areas in the set of images or simply to reject background that does not relate to tissue. Segmentation may involve any or all of the following image processing techniques:

1. Variance based analysis to identify the seed tissue areas

2. Adaptive thresholding

3. Morphological operations (e.g. blob analysis)

4. Contour identification

5. Contour merging based on proximity heuristic rules

6. Calculation of invariant image moments

7. Edge extraction (e.g. Sobel edge detection)

8. Curvature flow filtering

9. Histogram matching to eliminate intensity variations between serial sections

10. Multi-resolution rigid/affine image registration (gradient descent optimizer)

11. Non-rigid deformation/transformation

12. Superpixel clustering

It will also be understood that image processing steps of the above kind can be carried on the WSIs or on individual patches after patch extraction. In some cases, it may be useful to carry out the same type of image processing both before and after patch extraction, i.e. as CNN pre-processing and CNN post-processing respectively. That is, some image processing may be done on the WSI before patch extraction and other image processing may be done on a patch after its extraction from the WSI.

These image processing steps are described by way of example and should not be interpreted as being in any way limitative on the scope of the invention. For example, the CNN could work directly with color images if sufficient processing power is available.

Training & Prediction

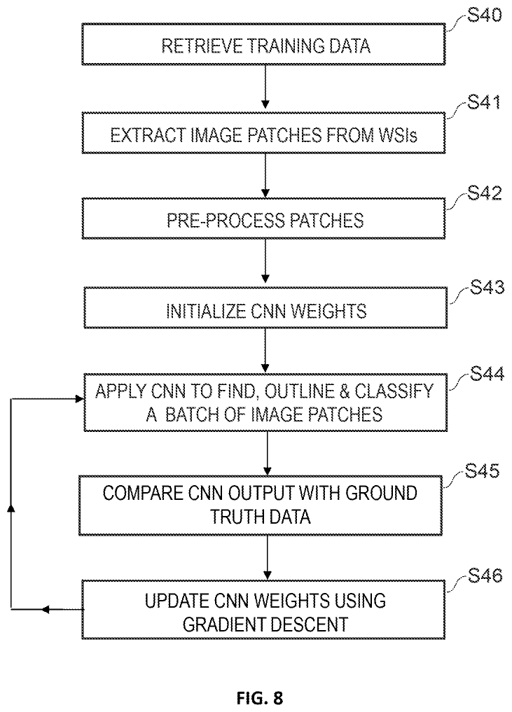

FIG. 8 is a flow diagram showing the steps involved in training the CNN.

In Step S40, training data is retrieved containing WSIs for processing which have been annotated by a clinician to find, outline and classify tumors. The clinician's annotations represent the ground truth data.

In Step S41, the WSIs are broken down into image patches, which are the input image patches for the CNN. That is, image patches are extracted from the WSI.

In Step S42, the image patches are pre-processed as described above. (Alternatively, or in addition, the WSIs could be pre-processed as described above prior to Step S41.)

In Step S43, initial values are set for the CNN weights, i.e. the weights between layers.

In Step S44, each of a batch of input image patches is input into the CNN and processed to find, outline and classify the patches on a pixel-by-pixel basis as described further above with reference to FIGS. 1A and 1B. The term outline here is not necessarily strictly technically the right term to use, since our method identifies each tumor (or tumor type) pixel, so it is perhaps more accurate to say that the CNN determines tumor areas for each tumor type.

In Step S45, the CNN output image patches are compared with the ground truth data. This may be done on a per-patch basis. Alternatively, if patches have been extracted that cover the entire WSI, then this may be done at the WSI level, or in sub-areas of the WSI made up of a contiguous batch of patches, e.g. one quadrant of the WSI. In such variants, the output image patches can be reassembled into a probability map for the entire WSI, or contiguous portion thereof, and the probability map can be compared with the ground truth data both by the computer and also by a user visually if the probability map is presented on the display as a semi-transparent overlay to the WSI, for example.

In Step S46, the CNN then learns from this comparison and updated the CNN weights, e.g. using a gradient descent approach. The learning is thus fed back into repeated processing of the training data as indicated in FIG. 8 by the return loop in the process flow, so that the CNN weights can be optimized.

After training, the CNN can be applied to WSIs independently of any ground truth data, i.e. in live use for prediction.

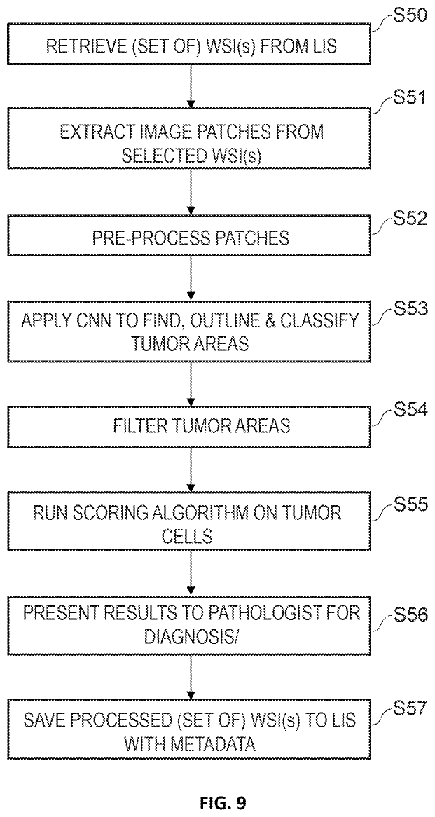

FIG. 9 is a flow diagram showing the steps involved in prediction using the CNN.

In Step S50, one or more WSIs are retrieved for processing, e.g. from a laboratory information system (LIS) or other histological data repository. The WSIs are pre-processed, for example as described above.

In Step S51, image patches are extracted from the or each WSI. The patches may cover the entire WSI or may be a random or non-random selection.

In Step S52, the image patches are pre-processed, for example as described above.

In Step S53, each of a batch of input image patches is input into the CNN and processed to find, outline and classify the patches on a pixel-by-pixel basis as described further above with reference to FIGS. 1A and 1B. The output patches can then be reassembled as a probability map for the WSI from which the input image patches were extracted. The probability map can be compared with the WSI both by the computer apparatus in digital processing and also by a user visually, if the probability map is presented on the display as a semi-transparent overlay on the WSI or alongside the WSI, for example.

In Step S54, the tumor areas are filtered excluding tumors that are likely to be false positives, for example areas that are too small or areas that may be edge artifacts.

In Step S55, a scoring algorithm is run. The scoring is cell specific and the score may be aggregated for each tumor, and/or further aggregated for the WSI (or sub-area of the WSI).

In Step S56, the results are presented to a pathologist or other relevantly skilled clinician for diagnosis, e.g. by display of the annotated WSI on a suitable high-resolution monitor.

In Step S57, the results of the CNN, i.e. the probability map data and optionally also metadata relating to the CNN parameters together with any additional diagnostic information added by the pathologist, are saved in a way that is linked to the patient data file containing the WSI, or set of WSIs, that have been processed by the CNN. The patient data file in the LIS or other histological data repository is thus supplemented with the CNN results.

Automated Determination of the Need for Additional Tests

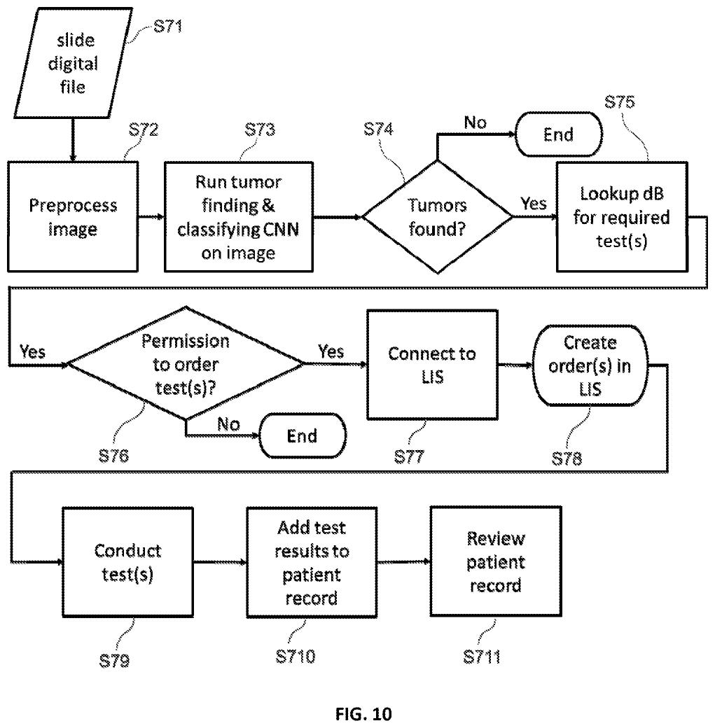

FIG. 10 is a flow diagram provided by a workflow control software according to an embodiment of the disclosure.

Step S71 provides an image data file containing image data of a WSI, as may have been generated by a slide scanner. It will be appreciated the image data file may include multiple images, e.g. one for each of a plurality of stains, or one for each of a different depth in the sample (a so-called z-stack) obtained by stepping the focal plane of the microscope through a transparent or semi-transparent sample of finite depth. In the context of the present workflow, and by way of example, the starting point is an image data file containing an H&E stain image, or possibly a z-stack of images from the same H&E slide. In other examples, the initial image or set of images provided in Step S71 could be from an unstained slide, or a slide stained with some other suitable stain or combination of stains.

Step S72 is an optional step where some image pre-processing may be performed, as described by way of example further above, such as variance-based analysis, adaptive thresholding, morphological operations and so forth.

Step S73 runs the above-described CNN, in particular as described with reference to Steps S51 to S54 of FIG. 9. A pixel-by-pixel classification of tissue type is performed to mark tumor pixels, followed optionally also by segmentation to outline tumors (i.e. tumor areas). The tissue type is a classification by carcinoma type. For the optional segmentation, it is generally the case that contiguous tumor pixels, i.e. ones that are touching each other or in close proximity to each other, belong to a common tumor. More complex segmentation criteria will however usually be included to improve reliability, e.g. to identify two touching tumors of different pixel classifications, e.g. associated with two different cancerous cell classifications. The CNN assigns each pixel a probability, which is the probability vector representing the probability of the pixel belonging to each of the N classes that the CNN has been trained to detect. For example, in the case of a CNN trained to distinguish between invasive, in situ and non-tumor areas, each pixel will be assigned a vector of length 3. A pixel at location k may have a probability vector [0.1, 0.2, 0.7] indicating there is a 10% probability the pixel is in an invasive area, a 20% probability it is in an in situ area and a 70% probability it is in a non-tumor area.

In Step S74, performed after Step S73 in which the above-described CNN has assigned probability vectors to each pixel in the image, a secondary algorithm based on conventional image processing techniques (e.g. as listed further above in connection with segmentation) computes the presence and abundance of pixels of different tumor types in each image. For example, a single pass can be made through the probability map, assigning each pixel to its class and computing the sums to make a count of pixels in each class is computed for the WSI. If the number of pixels in a class is above a pre-set significance threshold for that class, the tumor type represented by the class is considered to be present, i.e. present to a diagnostically significantly degree, in the tissue sample. The threshold can be specified in a number of different ways. For example, the threshold may be defined in absolute terms as a specific minimum area (or equivalent number of pixels), or in relative terms as a percentage of tissue in the WSI (i.e. ignoring non-tissue areas in the WSI). The subdivision of the WSI into tissue and non-tissue areas can be detected by a separate pre-processing algorithm which may be based on traditional image processing, or which may use a CNN that works in a similar fashion to that described above. Alternatively, the same CNN that classifies tissue into non-tumorous and multiple tumor types may also include a non-tissue class. The threshold may also be computed having regard to segmentation results, e.g. to ignore pixels that are not situated in tumors that are below a certain size, or to count all pixels in a tumor regardless of class if the tumor has a whole has been determined to be a tumor of a certain class through an aggregate computation of which tissue classes are present and in what absolute or relative abundance over the tumor area.

A more sophisticated approach to identifying whether a significance threshold has been exceeded for a given tissue class is one based on a multi-factorial scoring. For example, the data generated by the tumor-finding CNN in Step S73, i.e. the tumor-specific data, could be used to compute a set of summary statistics for each tumor as determined by segmentation. For example, for each tumor, a score may be computed as the mathematical average of the above-mentioned probability values for all the pixels contained in that tumor (area). Some other summary statistic such as median, weighted average, etc. may also be used to compute a score. The set of summary statistics may include for example dimensional or morphological attributes of a tumor, such as tumor area as measured by the number of pixels in the tumor or shape of tumor area or prevalence of a certain pixel classification such as invasive tumor and in situ tumor. Usually for each tumor the average and standard deviation of tumor probability, tumor area and length of the tumors' greatest dimension will be included. Tumor areas are not necessarily from a single slide; they may belong to separate slides, e.g. the tissue samples of two slides may be stained with different stains and thus highlight different classes of tumor cells, so that some tumors are identified in a first slide and other tumors in a second slide.

A still more sophisticated scoring approach could include other parameters derived from the CNN training data. For example, it is possible to predict patient risk using a CNN trained on a combination of histological image data (i.e. tumors identified in image data) and patient-specific genetic (i.e. genomic) data. A CNN of this kind is described in Mobadersany et al 2018.

In other implementations, the score can be computed using traditional image processing techniques applied to the tumors identified by the CNN. For example, shape and texture measures can be combined with genetic data to create a set of statistical measures to include in the summary statistics. The score may be based on a composite score indicating importance for patient survival, e.g. 5-year survival probability, or be a simple single parameter ranking, e.g. based on a size parameter of the tumor such as area or maximum dimension, or a morphological parameter such as roundness. A support vector machine or random forest algorithm may use these features to predict risk of metastasis. Either way a metastasis risk score will be computed and associated with each tumor area. We define the risk of metastasis as the probability that cells from this tumor will metastasize to other parts of the body. The significantly present tumor types identified in the WSI are returned as a list.

Instead of or as well as use of scoring to define whether a given tumor type is present in significant amounts, filtering of tumors may be applied to filter out any tumors deemed to be insignificant, e.g. very small tumors. The filtering may be based on the above-mentioned summary statistics, for example. The filter may choose to pass only tumors with a maximum dimension above a threshold value, e.g. 100 micrometers, that have an average probability above a threshold value, e.g. 50%.

If a tissue class of at least one tumor type has been identified in Step S74 as being present in clinically significant amounts, the process flow continues to Step S75.

In Step S75, each significantly present tumor type identified in the WSI, i.e. each tumor type in the list returned as the output of Step S74, is checked against a database which stores protocol definitions. Each protocol definition links tumor types to tests that should, or may, be performed on samples containing that tumor type, and optionally also to permissions that are required to order each such test. The database may be modified by a user with appropriate authority to add, delete or modify tests and/or to change permissions associated with test ordering, which may be a person or persons that have superuser or administrator rights. A database query based on applying a list as output from Step S74 returns a query result listing the additional tests and associated permissions that are required for furthering the analysis of the biopsy. An authorization for any particular test may be granted on an individual order basis, e.g. by a user with the necessary authority, or in a blanket fashion, which would allow the system to place orders for such a test without the need to fetch a specific user permission. The patient's profile, as accessible in or via a pointer in the patient record may also be referred to deduce a permission. A permission may also be implied from a user who is logged in currently according to that user's rights in the workflow control software.

If and when the necessary authorizations are present, e.g. provided by blanket permission for a particular test, or having been fetched from an appropriately authorized user, process flow proceeds to Step S77.

In Step S77, the workflow control software connects to the clinical network, e.g. the LIS, specifically to the patient record including the WSI processed in Steps S71 to S76.

In Step S78, the workflow control software creates and places orders for the tests output from Step S76. (Here the use of plural is for convenience and does not exclude the possibility there is only one test order.) For example, the system may use the Health Level-7 (HL7) protocol (Kush et al. 2008) to connect to the LIS and submit an order for that test.

In Step S79, the ordered tests are conducted. Conducting the orders may, for example, trigger extra manual laboratory work in applying one or more stains, e.g. protein-specific markers, to one or more serial sections of the section used for the H&E slide in order to prepare one or more new slides, followed by automated acquisition of a WSI for each new slide. In other examples, conducting an additional test may be fully automated and so performed under control of the workflow control software. The image acquisition may be followed by further automated image processing of the new slide's WSI, which may include conventional image processing and/or CNN-based image processing as appropriate. Moreover it will be understood that the image processing of each new slide's WSI is likely to be conducted jointly with reference to the H&E WSI and possibly also further new slide WSI's from other stains. For example, the different WSIs of the serial sections from the same biopsy may be composited with the aid of suitable warp transforms to generate a pixel mapping between the different WSIs.

In Step S710, the additional test results are added to the biopsy record containing the original, i.e. initial, H&E image, notably including the WSIs with the new stains and any subsequent image processing on those new WSIs.

In Step S711, the patient record, which now includes the additional tests identified, ordered and conducted in Steps S75 to S79, is loaded into the pathologist's workstation which has running thereon a pathology visualization application which is operable to generate a visualization of the slide image(s) and display each such image to the user in a graphical user interface (GUI) window of a display device that forms part of the workstation. Typically, the image displayed will either be in the form of a combined overlay view or a multi-tile view. In an overlay view the raw data (possibly processed) is displayed with the tissue type classification data (which typically will be integrated with the segmentation data) overlaid on top. The tissue type classification data and/or segmentation data may be translated for the visualization into a shading and/or outline of each tumor, e.g. the outline may represent the segmentation and the shading may use color or different kinds of hatching to represent different tissue classes. In the case of an overlay image in particular it will be beneficial for the visualization to incorporate such tissue-class or tumor-class specific shading and/or outlining. Non-tumorous areas of tissue may not be marked at all or may be shaded with a color wash of high transparency, e.g. a gray or blue wash. In a multi-tile view, what were the different layers in the overlay view are displayed side-by-side as tiles, so there will be a tile showing raw image data (possibly processed) and a tile showing the tissue type classification data and/or segmentation data of the filtered tumor areas. If desired, a separate tile may be displayed for each tissue-type or tumor-type classification. The visualization may also present tumors of a tissue class that has not been specifically tested with a stain specifically relevant to that tissue class differently from those that have been tested for with a specific stain, e.g. monochrome such as gray shading or outlines could be used for tumors that relate to tumor classes of non-tested types and respective colors for tumor classes of tested types.

In summary, a CNN processes a histological image to identify tumors by classifying image pixels as belonging to one of multiple tissue classes include one or more classes for tumorous tissue. The need for any follow-on tests is then determined based on tissue classes found in the image by the CNN and with reference to a tissue-class-specific protocol. For each such follow-on test decided upon, an order is automatically created and submitted within a laboratory information system. This automated workflow ensures that, at the time of first review by a pathologist, the patient record already includes not only a basic histological image (the one reviewed by the CNN), but also results from the additional tests automatically ordered as a result of the CNN analysis.

CNN Computing Platform

The proposed image processing may be carried out on a variety of computing architectures, in particular ones that are optimized for neural networks, which may be based on CPUs, GPUs, TPUs, FPGAs and/or ASICs. In some embodiments, the neural network is implemented using Google's Tensorflow software library running on Nvidia GPUs from Nvidia Corporation, Santa Clara, Calif., such as the Tesla K80 GPU. In other embodiments, the neural network can run on generic CPUs. Faster processing can be obtained by a purpose-designed processor for performing CNN calculations, for example the TPU disclosed in Jouppi et al 2017, the full contents of which is incorporated herein by reference.

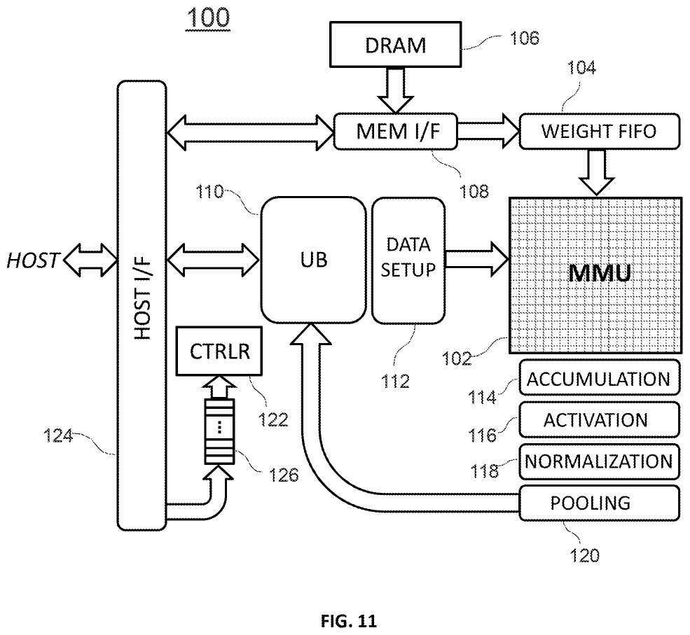

FIG. 11 shows the TPU of Jouppi et al 2017, being a simplified reproduction of Jouppi's FIG. 1. The TPU 100 has a systolic matrix multiplication unit (MMU) 102 which contains 256×256 MACs that can perform 8-bit multiply-and-adds on signed or unsigned integers. The weights for the MMU are supplied through a weight FIFO buffer 104 that in turn reads the weights from a memory 106, in the form of an off-chip 8 GB DRAM, via a suitable memory interface 108. A unified buffer (UB) 110 is provided to store the intermediate results. The MMU 102 is connected to receives inputs from the weight FIFO interface 104 and the UB 110 (via a systolic data setup unit 112) and outputs the 16-bit products of the MMU processing to an accumulator unit 114. An activation unit 116 performs nonlinear functions on the data held in the accumulator unit 114. After further processing by a normalizing unit 118 and a pooling unit 120, the intermediate results are sent to the UB 110 for resupply to the MMU 102 via the data setup unit 112. The pooling unit 120 may perform maximum pooling (i.e. maxpooling) or average pooling as desired. A programmable DMA controller 122 transfers data to or from the TPU's host computer and the UB 110. The TPU instructions are sent from the host computer to the controller 122 via a host interface 124 and an instruction buffer 126.

It will be understood that the computing power used for running the neural network, whether it be based on CPUs, GPUs or TPUs, may be hosted locally in a clinical network, e.g. the one described below, or remotely in a data center.

Network & Computing & Scanning Environment

The proposed computer-automated method operates in the context of a laboratory information system (LIS) which in turn is typically part of a larger clinical network environment, such as a hospital information system (HIS) or picture archiving and communication system (PACS). In the LIS, the WSIs will be retained in a database, typically a patient information database containing the electronic medical records of individual patients. The WSIs will be taken from stained tissue samples mounted on slides, the slides bearing printed barcode labels by which the WSIs are tagged with suitable metadata, since the microscopes acquiring the WSIs are equipped with barcode readers. From a hardware perspective, the LIS will be a conventional computer network, such as a local area network (LAN) with wired and wireless connections as desired.

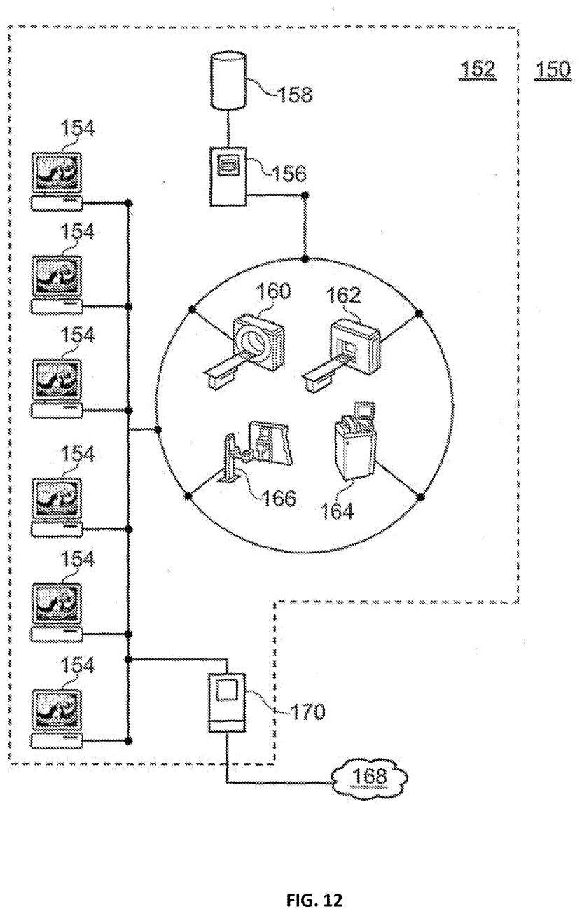

FIG. 12 shows an example computer network which can be used in conjunction with embodiments of the invention. The network 150 comprises a LAN in a hospital 152. The hospital 152 is equipped with a number of workstations 154 which each have access, via the local area network, to a hospital computer server 156 having an associated storage device 158. A LIS, HIS or PACS archive is stored on the storage device 158 so that data in the archive can be accessed from any of the workstations 154. One or more of the workstations 154 has access to a graphics card and to software for computer-implementation of methods of generating images as described hereinbefore. The software may be stored locally at the or each workstation 154 or may be stored remotely and downloaded over the network 150 to a workstation 154 when needed. In other example, methods embodying the invention may be executed on the computer server with the workstations 154 operating as terminals. For example, the workstations may be configured to receive user input defining a desired histological image data set and to display resulting images while CNN analysis is performed elsewhere in the system. Also, a number of histological and other medical imaging devices 160, 162, 164, 166 are connected to the hospital computer server 156. Image data collected with the devices 160, 162, 164, 166 can be stored directly into the LIS, HIS or PACS archive on the storage device 156. Thus, histological images can be viewed and processed immediately after the corresponding histological image data are recorded. The local area network is connected to the Internet 168 by a hospital Internet server 170, which allows remote access to the LIS, HIS or PACS archive. This is of use for remote accessing of the data and for transferring data between hospitals, for example, if a patient is moved, or to allow external research to be undertaken.

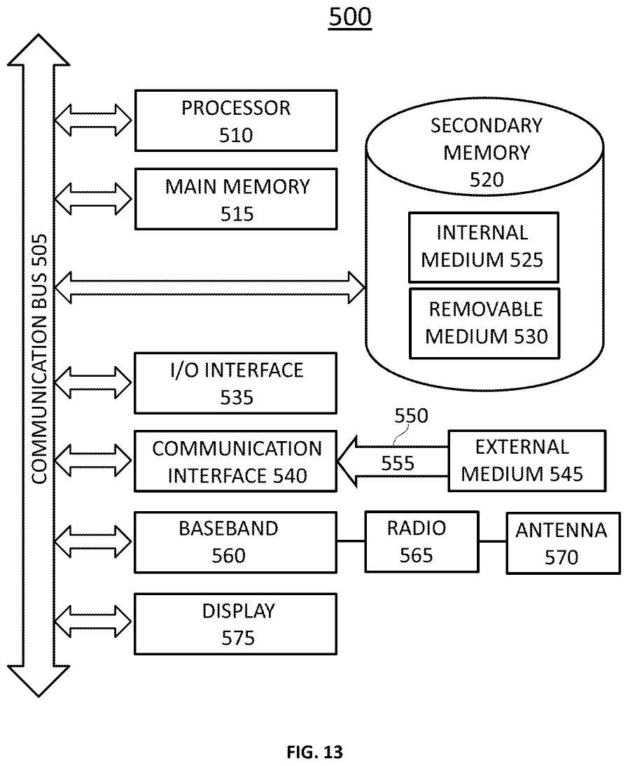

FIG. 13 is a block diagram illustrating an example computing apparatus 500 that may be used in connection with various embodiments described herein. For example, computing apparatus 500 may be used as a computing node in the above-mentioned LIS or PACS system, for example a host computer from which CNN processing is carried out in conjunction with a suitable GPU, or the TPU shown in FIG. 11.

Computing apparatus 500 can be a server or any conventional personal computer, or any other processor-enabled device that is capable of wired or wireless data communication. Other computing apparatus, systems and/or architectures may be also used, including devices that are not capable of wired or wireless data communication, as will be clear to those skilled in the art.

Computing apparatus 500 preferably includes one or more processors, such as processor 510. The processor 510 may be for example a CPU, GPU, TPU or arrays or combinations thereof such as CPU and TPU combinations or CPU and GPU combinations. Additional processors may be provided, such as an auxiliary processor to manage input/output, an auxiliary processor to perform floating point mathematical operations (e.g. a TPU), a special-purpose microprocessor having an architecture suitable for fast execution of signal processing algorithms (e.g., digital signal processor, image processor), a slave processor subordinate to the main processing system (e.g., back-end processor), an additional microprocessor or controller for dual or multiple processor systems, or a coprocessor. Such auxiliary processors may be discrete processors or may be integrated with the processor 510. Examples of CPUs which may be used with computing apparatus 500 are, the Pentium processor, Core i7 processor, and Xeon processor, all of which are available from Intel Corporation of Santa Clara, Calif. An example GPU which may be used with computing apparatus 500 is Tesla K80 GPU of Nvidia Corporation, Santa Clara, Calif.

Processor 510 is connected to a communication bus 505. Communication bus 505 may include a data channel for facilitating information transfer between storage and other peripheral components of computing apparatus 500. Communication bus 505 further may provide a set of signals used for communication with processor 510, including a data bus, address bus, and control bus (not shown). Communication bus 505 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, or standards promulgated by the Institute of Electrical and Electronics Engineers (IEEE) including IEEE 488 general-purpose interface bus (GPIB), IEEE 696/S-100, and the like.

Computing apparatus 500 preferably includes a main memory 515 and may also include a secondary memory 520. Main memory 515 provides storage of instructions and data for programs executing on processor 510, such as one or more of the functions and/or modules discussed above. It should be understood that computer readable program instructions stored in the memory and executed by processor 510 may be assembler instructions, instruction-set-architecture (ISA) instructions, machine instructions, machine dependent instructions, microcode, firmware instructions, state-setting data, configuration data for integrated circuitry, or either source code or object code written in and/or compiled from any combination of one or more programming languages, including without limitation Smalltalk, C/C++, Java, JavaScript, Perl, Visual Basic, .NET, and the like. Main memory 515 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Secondary memory 520 may optionally include an internal memory 525 and/or a removable medium 530. Removable medium 530 is read from and/or written to in any well-known manner. Removable storage medium 530 may be, for example, a magnetic tape drive, a compact disc (CD) drive, a digital versatile disc (DVD) drive, other optical drive, a flash memory drive, etc.

Removable storage medium 530 is a non-transitory computer-readable medium having stored thereon computer-executable code (i.e., software) and/or data. The computer software or data stored on removable storage medium 530 is read into computing apparatus 500 for execution by processor 510.

The secondary memory 520 may include other similar elements for allowing computer programs or other data or instructions to be loaded into computing apparatus 500. Such means may include, for example, an external storage medium 545 and a communication interface 540, which allows software and data to be transferred from external storage medium 545 to computing apparatus 500. Examples of external storage medium 545 may include an external hard disk drive, an external optical drive, an external magneto-optical drive, etc. Other examples of secondary memory 520 may include semiconductor-based memory such as programmable read-only memory (PROM), erasable programmable read-only memory (EPROM), electrically erasable read-only memory (EEPROM), or flash memory (block-oriented memory similar to EEPROM).

As mentioned above, computing apparatus 500 may include a communication interface 540. Communication interface 540 allows software and data to be transferred between computing apparatus 500 and external devices (e.g. printers), networks, or other information sources. For example, computer software or executable code may be transferred to computing apparatus 500 from a network server via communication interface 540. Examples of communication interface 540 include a built-in network adapter, network interface card (NIC), Personal Computer Memory Card International Association (PCMCIA) network card, card bus network adapter, wireless network adapter, Universal Serial Bus (USB) network adapter, modem, a network interface card (NIC), a wireless data card, a communications port, an infrared interface, an IEEE 1394 fire-wire, or any other device capable of interfacing system 550 with a network or another computing device.

Communication interface 540 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 540 are generally in the form of electrical communication signals 555. These signals 555 may be provided to communication interface 540 via a communication channel 550. In an embodiment, communication channel 550 may be a wired or wireless network, or any variety of other communication links. Communication channel 550 carries signals 555 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (i.e., computer programs or software) is stored in main memory 515 and/or the secondary memory 520. Computer programs can also be received via communication interface 540 and stored in main memory 515 and/or secondary memory 520. Such computer programs, when executed, enable computing apparatus 500 to perform the various functions of the disclosed embodiments as described elsewhere herein.

In this document, the term “computer-readable medium” is used to refer to any non-transitory computer-readable storage media used to provide computer-executable code (e.g., software and computer programs) to computing apparatus 500. Examples of such media include main memory 515, secondary memory 520 (including internal memory 525, removable medium 530, and external storage medium 545), and any peripheral device communicatively coupled with communication interface 540 (including a network information server or other network device). These non-transitory computer-readable media are means for providing executable code, programming instructions, and software to computing apparatus 500. In an embodiment that is implemented using software, the software may be stored on a computer-readable medium and loaded into computing apparatus 500 by way of removable medium 530, I/O interface 535, or communication interface 540. In such an embodiment, the software is loaded into computing apparatus 500 in the form of electrical communication signals 555. The software, when executed by processor 510, preferably causes processor 510 to perform the features and functions described elsewhere herein.

I/O interface 535 provides an interface between one or more components of computing apparatus 500 and one or more input and/or output devices. Example input devices include, without limitation, keyboards, touch screens or other touch-sensitive devices, biometric sensing devices, computer mice, trackballs, pen-based pointing devices, and the like. Examples of output devices include, without limitation, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), and the like.

Computing apparatus 500 also includes optional wireless communication components that facilitate wireless communication over a voice network and/or a data network. The wireless communication components comprise an antenna system 570, a radio system 565, and a baseband system 560. In computing apparatus 500, radio frequency (RF) signals are transmitted and received over the air by antenna system 570 under the management of radio system 565.

Antenna system 570 may comprise one or more antennae and one or more multiplexors (not shown) that perform a switching function to provide antenna system 570 with transmit and receive signal paths. In the receive path, received RF signals can be coupled from a multiplexor to a low noise amplifier (not shown) that amplifies the received RF signal and sends the amplified signal to radio system 565.

Radio system 565 may comprise one or more radios that are configured to communicate over various frequencies. In an embodiment, radio system 565 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 565 to baseband system 560.

If the received signal contains audio information, then baseband system 560 decodes the signal and converts it to an analog signal. Then the signal is amplified and sent to a speaker. Baseband system 560 also receives analog audio signals from a microphone. These analog audio signals are converted to digital signals and encoded by baseband system 560. Baseband system 560 also codes the digital signals for transmission and generates a baseband transmit audio signal that is routed to the modulator portion of radio system 565. The modulator mixes the baseband transmit audio signal with an RF carrier signal generating an RF transmit signal that is routed to antenna system 570 and may pass through a power amplifier (not shown). The power amplifier amplifies the RF transmit signal and routes it to antenna system 570 where the signal is switched to the antenna port for transmission.

Baseband system 560 is also communicatively coupled with processor 510, which may be a central processing unit (CPU). Processor 510 has access to data storage areas 515 and 520. Processor 510 is preferably configured to execute instructions (i.e., computer programs or software) that can be stored in main memory 515 or secondary memory 520. Computer programs can also be received from baseband processor 560 and stored in main memory 510 or in secondary memory 520 or executed upon receipt. Such computer programs, when executed, enable computing apparatus 500 to perform the various functions of the disclosed embodiments. For example, data storage areas 515 or 520 may include various software modules.

The computing apparatus further comprises a display 575 directly attached to the communication bus 505 which may be provided instead of or addition to any display connected to the I/O interface 535 referred to above.

Various embodiments may also be implemented primarily in hardware using, for example, components such as application specific integrated circuits (ASICs), programmable logic arrays (PLA), or field programmable gate arrays (FPGAs). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit, or step is for ease of description. Specific functions or steps can be moved from one module, block, or circuit to another without departing from the invention.

Moreover, the various illustrative logical blocks, modules, functions, and methods described in connection with the embodiments disclosed herein can be implemented or performed with a general purpose processor, a digital signal processor (DSP), an ASIC, FPGA, or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such that the processor can read information from, and write information to, the storage medium. In the alternative, the storage medium can be integral to the processor. The processor and the storage medium can also reside in an ASIC.

A computer readable storage medium, as referred to herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

Embodiments of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The illustrated flowcharts and block diagrams illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Apparatus and methods embodying the invention are capable of being hosted in and delivered by a cloud computing environment. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

It will be clear to one skilled in the art that many improvements and modifications can be made to the foregoing exemplary embodiment without departing from the scope of the present disclosure.

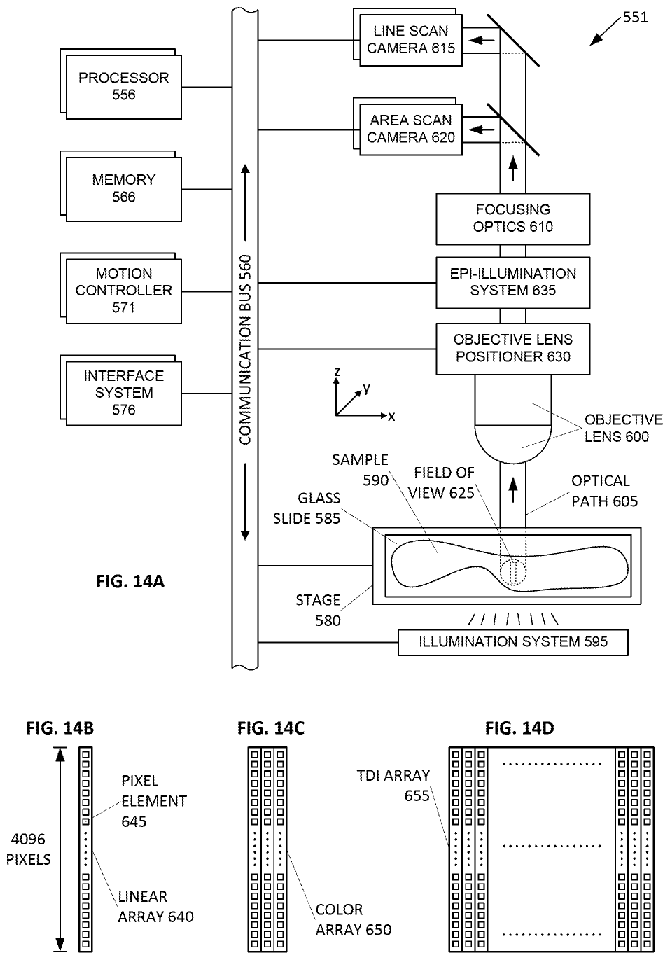

FIG. 14A is a block diagram illustrating an example processor enabled device 551 that may be used in connection with various embodiments described herein. Alternative forms of the device 551 may also be used as will be understood by the skilled artisan. In the illustrated embodiment, the device 551 is presented as a digital imaging device (also referred to herein as a scanner system or a scanning system) that comprises one or more processors 556, one or more memories 566, one or more motion controllers 571, one or more interface systems 576, one or more movable stages 580 that each support one or more glass slides 585 with one or more samples 590, one or more illumination systems 595 that illuminate the sample, one or more objective lenses 600 that each define an optical path 605 that travels along an optical axis, one or more objective lens positioners 630, one or more optional epi-illumination systems 635 (e.g., included in a fluorescence scanner system), one or more focusing optics 610, one or more line scan cameras 615 and/or one or more area scan cameras 620, each of which define a separate field of view 625 on the sample 590 and/or glass slide 585. The various elements of the scanner system 551 are communicatively coupled via one or more communication busses 560. Although there may be one or more of each of the various elements of the scanner system 551, for simplicity in the description that follows, these elements will be described in the singular except when needed to be described in the plural to convey the appropriate information.

The one or more processors 556 may include, for example, a central processing unit (“CPU”) and a separate graphics processing unit (“GPU”) capable of processing instructions in parallel or the one or more processors 556 may include a multicore processor capable of processing instructions in parallel. Additional separate processors may also be provided to control particular components or perform particular functions such as image processing. For example, additional processors may include an auxiliary processor to manage data input, an auxiliary processor to perform floating point mathematical operations, a special-purpose processor having an architecture suitable for fast execution of signal processing algorithms (e.g., digital signal processor), a slave processor subordinate to the main processor (e.g., back-end processor), an additional processor for controlling the line scan camera 615, the stage 580, the objective lens 225, and/or a display (not shown). Such additional processors may be separate discrete processors or may be integrated with the processor 556.

The memory 566 provides storage of data and instructions for programs that can be executed by the processor 556. The memory 566 may include one or more volatile and persistent computer-readable storage mediums that store the data and instructions, for example, a random access memory, a read only memory, a hard disk drive, removable storage drive, and the like. The processor 556 is configured to execute instructions that are stored in memory 566 and communicate via communication bus 560 with the various elements of the scanner system 551 to carry out the overall function of the scanner system 551.

The one or more communication busses 560 may include a communication bus 560 that is configured to convey analog electrical signals and may include a communication bus 560 that is configured to convey digital data. Accordingly, communications from the processor 556, the motion controller 571, and/or the interface system 576 via the one or more communication busses 560 may include both electrical signals and digital data. The processor 556, the motion controller 571, and/or the interface system 576 may also be configured to communicate with one or more of the various elements of the scanning system 551 via a wireless communication link.

The motion control system 571 is configured to precisely control and coordinate XYZ movement of the stage 580 and the objective lens 600 (e.g., via the objective lens positioner 630). The motion control system 571 is also configured to control movement of any other moving part in the scanner system 551. For example, in a fluorescence scanner embodiment, the motion control system 571 is configured to coordinate movement of optical filters and the like in the epi-illumination system 635.

The interface system 576 allows the scanner system 551 to interface with other systems and human operators. For example, the interface system 576 may include a user interface to provide information directly to an operator and/or to allow direct input from an operator. The interface system 576 is also configured to facilitate communication and data transfer between the scanning system 551 and one or more external devices that are directly connected (e.g., a printer, removable storage medium) or external devices such as an image server system, an operator station, a user station, and an administrative server system that are connected to the scanner system 551 via a network (not shown).

The illumination system 595 is configured to illuminate a portion of the sample 590. The illumination system may include, for example, a light source and illumination optics. The light source could be a variable intensity halogen light source with a concave reflective mirror to maximize light output and a KG-1 filter to suppress heat. The light source could also be any type of arc-lamp, laser, or other source of light. In one embodiment, the illumination system 595 illuminates the sample 590 in transmission mode such that the line scan camera 615 and/or area scan camera 620 sense optical energy that is transmitted through the sample 590. Alternatively, or in combination, the illumination system 595 may also be configured to illuminate the sample 590 in reflection mode such that the line scan camera 615 and/or area scan camera 620 sense optical energy that is reflected from the sample 590. Overall, the illumination system 595 is configured to be suitable for interrogation of the microscopic sample 590 in any known mode of optical microscopy.

In one embodiment, the scanner system 551 optionally includes an epi-illumination system 635 to optimize the scanner system 551 for fluorescence scanning. Fluorescence scanning is the scanning of samples 590 that include fluorescence molecules, which are photon sensitive molecules that can absorb light at a specific wavelength (excitation). These photon sensitive molecules also emit light at a higher wavelength (emission). Because the efficiency of this photoluminescence phenomenon is very low, the amount of emitted light is often very low. This low amount of emitted light typically frustrates conventional techniques for scanning and digitizing the sample 590 (e.g., transmission mode microscopy). Advantageously, in an optional fluorescence scanner system embodiment of the scanner system 551, use of a line scan camera 615 that includes multiple linear sensor arrays (e.g., a time delay integration (“TDI”) line scan camera) increases the sensitivity to light of the line scan camera by exposing the same area of the sample 590 to each of the multiple linear sensor arrays of the line scan camera 615. This is particularly useful when scanning faint fluorescence samples with low emitted light.

Accordingly, in a fluorescence scanner system embodiment, the line scan camera 615 is preferably a monochrome TDI line scan camera. Advantageously, monochrome images are ideal in fluorescence microscopy because they provide a more accurate representation of the actual signals from the various channels present on the sample. As will be understood by those skilled in the art, a fluorescence sample 590 can be labeled with multiple florescence dyes that emit light at different wavelengths, which are also referred to as “channels.”

Furthermore, because the low and high end signal levels of various fluorescence samples present a wide spectrum of wavelengths for the line scan camera 615 to sense, it is desirable for the low and high end signal levels that the line scan camera 615 can sense to be similarly wide. Accordingly, in a fluorescence scanner embodiment, a line scan camera 615 used in the fluorescence scanning system 551 is a monochrome 10 bit 64 linear array TDI line scan camera. It should be noted that a variety of bit depths for the line scan camera 615 can be employed for use with a fluorescence scanner embodiment of the scanning system 551.

The movable stage 580 is configured for precise XY movement under control of the processor 556 or the motion controller 571. The movable stage may also be configured for movement in Z under control of the processor 556 or the motion controller 571. The moveable stage is configured to position the sample in a desired location during image data capture by the line scan camera 615 and/or the area scan camera. The moveable stage is also configured to accelerate the sample 590 in a scanning direction to a substantially constant velocity and then maintain the substantially constant velocity during image data capture by the line scan camera 615. In one embodiment, the scanner system 551 may employ a high precision and tightly coordinated XY grid to aid in the location of the sample 590 on the movable stage 580. In one embodiment, the movable stage 580 is a linear motor based XY stage with high precision encoders employed on both the X and the Y axis. For example, very precise nanometer encoders can be used on the axis in the scanning direction and on the axis that is in the direction perpendicular to the scanning direction and on the same plane as the scanning direction. The stage is also configured to support the glass slide 585 upon which the sample 590 is disposed.

The sample 590 can be anything that may be interrogated by optical microscopy. For example, a glass microscope slide 585 is frequently used as a viewing substrate for specimens that include tissues and cells, chromosomes, DNA, protein, blood, bone marrow, urine, bacteria, beads, biopsy materials, or any other type of biological material or substance that is either dead or alive, stained or unstained, labeled or unlabeled. The sample 590 may also be an array of any type of DNA or DNA-related material such as cDNA or RNA or protein that is deposited on any type of slide or other substrate, including any and all samples commonly known as a microarrays. The sample 590 may be a microtiter plate, for example a 96-well plate. Other examples of the sample 590 include integrated circuit boards, electrophoresis records, petri dishes, film, semiconductor materials, forensic materials, or machined parts.

Objective lens 600 is mounted on the objective positioner 630 which, in one embodiment, may employ a very precise linear motor to move the objective lens 600 along the optical axis defined by the objective lens 600. For example, the linear motor of the objective lens positioner 630 may include a 50 nanometer encoder. The relative positions of the stage 580 and the objective lens 600 in XYZ axes are coordinated and controlled in a closed loop manner using motion controller 571 under the control of the processor 556 that employs memory 566 for storing information and instructions, including the computer-executable programmed steps for overall scanning system 551 operation.

In one embodiment, the objective lens 600 is a plan apochromatic (“APO”) infinity corrected objective with a numerical aperture corresponding to the highest spatial resolution desirable, where the objective lens 600 is suitable for transmission mode illumination microscopy, reflection mode illumination microscopy, and/or epi-illumination mode fluorescence microscopy (e.g., an Olympus 40×, 0.75NA or 20×, 0.75 NA). Advantageously, objective lens 600 is capable of correcting for chromatic and spherical aberrations. Because objective lens 600 is infinity corrected, focusing optics 610 can be placed in the optical path 605 above the objective lens 600 where the light beam passing through the objective lens becomes a collimated light beam. The focusing optics 610 focus the optical signal captured by the objective lens 600 onto the light-responsive elements of the line scan camera 615 and/or the area scan camera 620 and may include optical components such as filters, magnification changer lenses, etc. The objective lens 600 combined with focusing optics 610 provides the total magnification for the scanning system 551. In one embodiment, the focusing optics 610 may contain a tube lens and an optional 2× magnification changer. Advantageously, the 2× magnification changer allows a native 20× objective lens 600 to scan the sample 590 at 40× magnification.

The line scan camera 615 comprises at least one linear array of picture elements (“pixels”). The line scan camera may be monochrome or color. Color line scan cameras typically have at least three linear arrays, while monochrome line scan cameras may have a single linear array or plural linear arrays. Any type of singular or plural linear array, whether packaged as part of a camera or custom-integrated into an imaging electronic module, can also be used. For example, 3 linear array (“red-green-blue” or “RGB”) color line scan camera or a 96 linear array monochrome TDI may also be used. TDI line scan cameras typically provide a substantially better signal-to-noise ratio (“SNR”) in the output signal by summing intensity data from previously imaged regions of a specimen, yielding an increase in the SNR that is in proportion to the square-root of the number of integration stages. TDI line scan cameras comprise multiple linear arrays, for example, TDI line scan cameras are available with 24, 32, 48, 64, 96, or even more linear arrays. The scanner system 551 also supports linear arrays that are manufactured in a variety of formats including some with 512 pixels, some with 1024 pixels, and others having as many as 4096 pixels. Similarly, linear arrays with a variety of pixel sizes can also be used in the scanner system 551. The salient requirement for the selection of any type of line scan camera 615 is that the motion of the stage 580 can be synchronized with the line rate of the line scan camera 615 so that the stage 580 can be in motion with respect to the line scan camera 615 during the digital image capture of the sample 590.

The image data generated by the line scan camera 615 is stored a portion of the memory 566 and processed by the processor 556 to generate a contiguous digital image of at least a portion of the sample 590. The contiguous digital image can be further processed by the processor 556 and the revised contiguous digital image can also be stored in the memory 566.

In an embodiment with two or more line scan cameras 615, at least one of the line scan cameras 615 can be configured to function as a focusing sensor that operates in combination with at least one of the line scan cameras 615 that is configured to function as an imaging sensor. The focusing sensor can be logically positioned on the same optical axis as the imaging sensor or the focusing sensor may be logically positioned before or after the imaging sensor with respect to the scanning direction of the scanner system 551. In such an embodiment with at least one line scan camera 615 functioning as a focusing sensor, the image data generated by the focusing sensor is stored in a portion of the memory 566 and processed by the one or more processors 556 to generate focus information to allow the scanner system 551 to adjust the relative distance between the sample 590 and the objective lens 600 to maintain focus on the sample during scanning. Additionally, in one embodiment the at least one line scan camera 615 functioning as a focusing sensor may be oriented such that each of a plurality of individual pixels of the focusing sensor is positioned at a different logical height along the optical path 605.

In operation, the various components of the scanner system 551 and the programmed modules stored in memory 566 enable automatic scanning and digitizing of the sample 590, which is disposed on a glass slide 585. The glass slide 585 is securely placed on the movable stage 580 of the scanner system 551 for scanning the sample 590. Under control of the processor 556, the movable stage 580 accelerates the sample 590 to a substantially constant velocity for sensing by the line scan camera 615, where the speed of the stage is synchronized with the line rate of the line scan camera 615. After scanning a stripe of image data, the movable stage 580 decelerates and brings the sample 590 to a substantially complete stop. The movable stage 580 then moves orthogonal to the scanning direction to position the sample 590 for scanning of a subsequent stripe of image data, e.g., an adjacent stripe. Additional stripes are subsequently scanned until an entire portion of the sample 590 or the entire sample 590 is scanned.

For example, during digital scanning of the sample 590, a contiguous digital image of the sample 590 is acquired as a plurality of contiguous fields of view that are combined together to form an image strip. A plurality of adjacent image strips are similarly combined together to form a contiguous digital image of a portion or the entire sample 590. The scanning of the sample 590 may include acquiring vertical image strips or horizontal image strips. The scanning of the sample 590 may be either top-to-bottom, bottom-to-top, or both (bi-directional) and may start at any point on the sample. Alternatively, the scanning of the sample 590 may be either left-to-right, right-to-left, or both (bi-directional) and may start at any point on the sample. Additionally, it is not necessary that image strips be acquired in an adjacent or contiguous manner. Furthermore, the resulting image of the sample 590 may be an image of the entire sample 590 or only a portion of the sample 590.

In one embodiment, computer-executable instructions (e.g., programmed modules and software) are stored in the memory 566 and, when executed, enable the scanning system 551 to perform the various functions described herein. In this description, the term “computer-readable storage medium” is used to refer to any media used to store and provide computer executable instructions to the scanning system 551 for execution by the processor 556. Examples of these media include memory 566 and any removable or external storage medium (not shown) communicatively coupled with the scanning system 551 either directly or indirectly, for example via a network (not shown).

FIG. 14B illustrates a line scan camera having a single linear array 640, which may be implemented as a charge coupled device (“CCD”) array. The single linear array 640 comprises a plurality of individual pixels 645. In the illustrated embodiment, the single linear array 640 has 4096 pixels. In alternative embodiments, linear array 640 may have more or fewer pixels. For example, common formats of linear arrays include 512, 1024, and 4096 pixels. The pixels 645 are arranged in a linear fashion to define a field of view 625 for the linear array 640. The size of the field of view varies in accordance with the magnification of the scanner system 551.

FIG. 14C illustrates a line scan camera having three linear arrays, each of which may be implemented as a CCD array. The three linear arrays combine to form a color array 650. In one embodiment, each individual linear array in the color array 650 detects a different color intensity, for example red, green, or blue. The color image data from each individual linear array in the color array 650 is combined to form a single field of view 625 of color image data.

FIG. 14D illustrates a line scan camera having a plurality of linear arrays, each of which may be implemented as a CCD array. The plurality of linear arrays combine to form a TDI array 655. Advantageously, a TDI line scan camera may provide a substantially better SNR in its output signal by summing intensity data from previously imaged regions of a specimen, yielding an increase in the SNR that is in proportion to the square-root of the number of linear arrays (also referred to as integration stages). A TDI line scan camera may comprise a larger variety of numbers of linear arrays, for example common formats of TDI line scan cameras include 24, 32, 48, 64, 96, 120 and even more linear arrays.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1-43. (canceled)

44. An apparatus, comprising:
a memory configured to store computer-executable instructions; and
a hardware processor in communication with the memory, wherein the computer-executable instructions, when executed by the processor, configure the processor to:
receive a histological image from a patient record;
generate, using a convolution neural network, an output image mapped to the histological image, the output image having one of a plurality of tissue classes assigned to each pixel of the output image, the convolution neural network being trained based on a training set data including (a) histological images and (b) ground truth data of tissue classes assigned to each pixel of the histological images, wherein the plurality of tissue classes includes at least one class representing non-tumorous tissue and at least one class representing tumorous tissue;
determine, for each tissue class in the output image, whether one or more tests should be performed on the tissue sample based on a protocol for that tissue class; and
in response to determining one or more tests should be performed, generate and transmit an order for each test to be performed.

45. The apparatus of claim 44, wherein the hardware processor is configured to determine whether one whether one or more tests should be performed comprises transmitting a query to a database organized to store protocols which specify tests to be performed, the query containing at least one of the tissue classes assigned to the output image.

46. The apparatus of claim 44, further comprising a data repository configured to store records of patient data including histological images.

47. The apparatus of claim 14, wherein the histological image is an H&E (hematoxylin and eosin) image.

48. The apparatus of claim 44, wherein the computer-executable instructions, when executed by the processor, further configure the processor to determine, for each tissue class, with reference to a stored protocol for that tissue class, whether any further tests should be performed on the tissue sample.

49. The apparatus of claim 48, wherein the computer-executable instructions, when executed by the processor, further configure the processor to transmit an order for each further test that is to be performed.

50. The apparatus of claim 49, wherein the computer-executable instructions, when executed by the processor, further configure the processor to generate the order for each further test that is to be performed.

51. The apparatus of claim 44, wherein the tissue classes include at least a first class for invasive tumors and a second class for in situ tumors.

52. The apparatus of claim 44, wherein the tissue classes include a tissue class for non-tumorous tissue.

53. The apparatus of claim 44, wherein the tissue classes include a tissue class representing areas where no tissue is identified.

54. A non-transitory computer readable medium for processing data of a tissue sample, the computer readable medium having program instructions for causing a hardware processor to perform a method of: comprising:
receive a histological image from a patient record;
generate, using a convolution neural network, an output image mapped to the histological image, the output image having one of a plurality of tissue classes assigned to each pixel of the output image, the convolution neural network being trained based on a training set data including (a) histological images and (b) ground truth data of tissue classes assigned to each pixel of the histological images, wherein the plurality of tissue classes includes at least one class representing non-tumorous tissue and at east one class representing tumorous tissue;
determine, for each tissue class in the output image, whether one or more tests should be performed on the tissue sample based on a protocol for that tissue class; and
response to determining one or more tests should be performed, generate and transmit an order for each test to be performed.

55. The computer readable medium of claim 54, wherein determine whether one or more tests should be performed comprises transmitting a query to a database organized to store protocols which specify tests to be performed, the query containing at least one of the tissue classes assigned to the output image.

56. The computer readable medium of claim 54, wherein the computer-executable instructions, when executed by the processor, further configure the processor to determine, for each tissue class, with reference to a stored protocol for that tissue class, whether any further tests should be performed on the tissue sample.

57. The computer readable medium of claim 54, wherein the computer-executable instructions, when executed by the processor, further configure the processor to transmit an order for each further test that is to be performed.

58. The computer readable medium of claim 57, wherein the computer-executable instructions, when executed by the processor, further configure the processor to generate the order for each further test that is to be performed.

59. The computer readable medium of claim 54, wherein the histological image is an H&E (hematoxylin and eosin) image.

60. The computer readable medium of claim 54, wherein the tissue classes includes a tissue class for invasive tumors, a second tissue class for in situ tumors, a tissue class for non-tumorous tissue, and a tissue class representing areas where no tissue is identified.

61. A method for processing data of a tissue sample, the method comprising:
receiving a histological image from a patient record;
generating, using a convolution neural network, an output image mapped to the histological image, the output image having one of a plurality of tissue classes assigned to each pixel of the output image, the convolution neural network being trained based on a training set data including (a) histological images and (b) ground truth data of tissue classes assigned to each pixel of the histological images, wherein the plurality of tissue classes includes at least one class representing non-tumorous tissue and at least one class representing tumorous tissue;
determining, for each tissue class in the output image, whether one or more tests should be performed on the tissue sample based on a protocol for that tissue class; and
in response to determining one or more tests should be performed, generating and transmitting an order for each test to be performed.

62. The method of claim 61, wherein determining whether one or more tests should be performed comprises submitting a query to a database organized to store protocols which specify tests to be performed, the query containing at least one of the tissue classes assigned to the output image.

63. The method of claim 61, further comprising determining, for each tissue class, with reference to a stored protocol for that tissue class, whether any further tests should be performed on the tissue sample, generating an order for each further test that is to be performed and transmitting an order for each further test that is to be performed.
